(19) United States
(12) Patent Application Publication
(10) Pub. No.: US 2014/0243562 A1
(43) Pub. Date: Aug. 28, 2014

(54) HYDROGENOLYSIS OF FURFURYL ALCOHOL TO 1,2-PENTANEDIOL

(71) Applicants: Marianne OMEIS, Dorsten (DE); Manfred NEUMANN, Marl (DE); Volker BREHME, Nottuln (DE); Christoph THEIS, Niederkassel (DE); Dorit WOLF, Oberursel (DE); Peter CLAUS, Berlin (DE); Martin LUCAS, Darmstadt (DE); Rene ECKERT, Griesheim (DE)

(72) Inventors: Marianne OMEIS, Dorsten (DE); Manfred NEUMANN, Marl (DE); Volker BREHME, Nottuln (DE); Christoph THEIS, Niederkassel (DE); Dorit WOLF, Oberursel (DE); Peter CLAUS, Berlin (DE); Martin LUCAS, Darmstadt (DE); Rene ECKERT, Griesheim (DE)

(73) Assignee: Evonik Industries AG, Essen (DE)

(21) Appl. No.: 14/088,721

(22) Filed: Nov. 25, 2013

(30) Foreign Application Priority Data
Feb. 28, 2013 (DE) 102013203420.2

(51) Int. Cl. C07C 29/132; C07D 307/12; B01J 23/46

(52) U.S. Cl. CPC C07C 29/132; B01J 23/462; C07D 307/12
USPC 568/865; 502/332; 549/497

(57) ABSTRACT

The present invention provides a process for preparing 1,2-pentanediol by reacting furfuryl alcohol with hydrogen in the presence of a catalyst system. The catalyst system contains platinum oxide or contains ruthenium supported on aluminum oxide or activated carbon. The invention also relates to the respective catalysts and processes for producing the catalyst system.

### CROSS REFERENCE TO RELATED APPLICATIONS

This application claims priority to German Application No. 102013203420.2, filed Feb. 28, 2013, the disclosure of which is incorporated herein by reference in its entirety.

### FIELD OF THE INVENTION

The present invention relates to a process for preparing 1,2-pentanediol, which is characterized in that furfuryl alcohol is reacted with hydrogen in the presence of a catalyst system. The various catalyst systems are heterogeneous catalysts. The catalyst system is, in a first aspect of the invention, ruthenium supported on a support composed of aluminum oxide.

In a second aspect of the invention, the catalyst system is ruthenium supported on a support composed of activated carbon. In a third aspect of the invention, the catalyst system is Pt(IV) oxide.

The invention additionally relates to processes for producing a catalyst system, where the catalyst system comprises ruthenium supported on a support composed of aluminum oxide or activated carbon. The invention also relates to the catalysts produced by the process for producing the catalyst system.

The process of the invention for preparing 1,2-pentanediol can be carried out according to the literature in a batch reactor, in a multibatch reactor or in a glass apparatus.

The present invention describes the most important by-products and a comprehensive reaction scheme for the hydrogenolysis of furfuryl alcohol over supported ruthenium catalysts, which also apply in the case of supported Pt catalysts.

The present invention provides, in a preferred embodiment, a process for preparing 1,2-pentanediol, which is characterized in that furfuryl alcohol is reacted with hydrogen in the presence of a catalyst system comprising ruthenium supported on a support composed of aluminum oxide. In preferred embodiments, the present invention describes, in particular, the influence of temperature, pressure, catalyst composition, solvent, pH, stirring speed and starting material concentration on the product distribution. In addition, the present invention describes the preparation of supported bimetallic Ru-Me catalysts and their performance in the process of the invention. In addition, the present invention describes the influence of small amounts of ionic liquids according to the SCILL concept. The conversions and selectivities were determined by analysing the liquid samples of the reaction mixtures by means of GC and GC-MS.

### BACKGROUND OF THE INVENTION

The major part of the chemical industry is at the present time based on fossil raw materials. The most important role is played by petroleum from which the hydrocarbons which as raw materials form the basis for the production of chemical intermediates and end products are obtained. The increasing demand for fossil fuels, especially in developing economies such as India or China, will in the next decades lead to a shortage of petroleum. [1] (The respective references are indicated in the literature index). In view of this background, renewable raw materials are a promising alternative for the chemical industry. In Germany, the term “renewable raw materials” was coined in 1973 in the context of the oil crisis as the negative impacts of the dependence on imported petroleum became particularly clear. Now, investment in renewable raw materials is also increasingly promoted by governmental bodies [2, 3] and international organizations such as SUSCHEM [4]. It is expected that as early as 2025, 30% of all chemicals will be produced from biomass in Europe. [5] Nature produces about 170 billion tonnes per annum of biomass by photosynthesis, of which only 3-4% is utilized by mankind. Carbohydrates form the lion's share at 75%. [6] They occur mostly in the form of polysaccharides which are composed of C5- or C6-monosaccharides. The polysaccharides hemicellulose (C5) and cellulose (C6) form, by incorporation of the phenolic biopolymer lignin, lignocellulose which is the structural framework of all woody plants. It is obtained in large quantities as waste product in agriculture and forestry. Furfural (for structural formula, see Scheme 1) is a chemical intermediate which is produced industrially exclusively from such biological waste. [7] It is at present the only unsaturated bulk chemical produced from carbohydrates. [8]

The utilization of renewable raw materials plays an important role in Green Chemistry, a philosophy which has steadily increased in importance since the 1990s and strives for a sustainable and safe chemical industry. [9], [55], [56] A further cornerstone of green chemistry is catalysis. The use of very active and selective catalysts offers a series of advantages which are important for sustainable chemistry: the use of stoichiometric reagents is avoided, the energy demand is reduced by reducing the reaction temperature and the formation of by-products requiring disposal is reduced. In this context, heterogeneous catalysts are particularly attractive since they can generally be separated off easily from the product without large quantities of energy having to be consumed for thermal separation processes. [10]

Furfural was isolated for the first time in 1831 by the German Chemist Wolfgang Dobereiner as by-product in the synthesis of formic acid when he treated carbohydrates with sulphuric acid and MnO₂. [11] The structural formula was elucidated in 1901 by Carl Harries. The first commercial production of furfural occurred in 1922 from oat husks. [7] At the present time, biological wastes obtained, for example, in the growing of sugar and maize serve as starting material for furfural production. These contain hemicellulose which is present as a constituent of lignocellulose in the cell walls of most plants. Hemicellulose is a mixture of polysaccharides which has a variable composition and contains mainly pentoses (simple sugars having five carbon atoms) as monomers. Treatment with hot sulphuric acid results in hydrolysis of hemicellulose and liberation of the monosaccharides. Under these reaction conditions, dehydration of the pentoses to furfural occurs as subsequent reaction. In 2003, 200 000 tonnes of furfural were produced worldwide, over half of it in China. About two thirds of the furfural produced are used for preparing furfuryl alcohol. [7, 12] The hydrogenation of furfural to furfuryl alcohol is carried out industrially over copper chromite catalysts and can be carried out both in the liquid phase and in the gas phase. [13, 14] The most important field of application of furfuryl alcohol is the production of foundry binders. In addition, it is employed, inter alia, in the modification of wood and as starting material for the synthesis of fine chemicals. [7, 15]

1,2-Pentanediol (for structural formula, see Scheme 1), also referred to as pentylene glycol, is a chiral diol having a stereogenic centre. The racemate is usually prepared by treating a mixture of 1-pentene and formic acid with hydrogen peroxide. [16] Synthesis of the enantiomers can be effected, for example, by use of chiral transition metal complexes as catalysts. [17] Like other 1,2-diols, pentylene glycol displays a strong antibacterial effect. It is employed, inter alia, in the synthesis of pesticides, crop protection agents and pharmaceuticals. [16]

Conventionally 1,2-pentanediol may be obtained by the hydrogenolysis of tetrahydrofurfuryl alcohol or by the hydrogenolysis of furfuryl alcohol.

A number of different heterogeneous catalysts have been described for the hydrogenolysis of furfuryl alcohol. Apart from copper and nickel catalysts, mainly platinum and ruthenium catalysts are conventionally used.

The platinum catalysts used can be divided into Pt(IV) oxide catalysts and supported platinum catalysts.

The Adams catalyst, named after its developer Roger Adams, is a popular catalyst for hydrogenation and hydrogenolysis reactions. It consists of platinum(IV) oxide which is reduced in situ to fine platinum powder (“platinum black”), the actual active catalyst. [18], [57] Adams reacted furfural over this at room temperature and a hydrogen pressure of 1-2 bar in ethanol with addition of iron chloride. He obtained a product mixture of tetrahydrofurfuryl alcohol (THFFOH, Y=35%), 1,2-pentanediol (1,2-PD, Y=20%), 1,5-pentanediol (1,5-PD, Y=8%) and 1-pentanol (1-POH, Y=11%). Adams was able to show that furfural is firstly fully hydrogenated using one equivalent of hydrogen to furfuryl alcohol (FFOH) which subsequently reacts to form the four abovementioned products. Without addition of iron chloride, the Adams catalyst displayed an extremely low catalytic activity. [19].

Nishimura used furfuryl alcohol as starting material. The hydrogenolysis was carried out at room temperature and atmospheric pressure in ethanol with additions of small amounts of 3 M hydrochloric acid. He obtained a product mixture consisting of pentane (Y=0.8%), 2-methyltetrahydrofuran (2-MTHF, Y=14.8%), 2-pentanol (2-POH, Y=5.5%), 1-pentanol (Y=11.6%), tetrahydrofurfuryl alcohol (Y=20.6%), 1,2-pentanediol (Y=13.3%) and 1,5-pentanediol (Y=3.3%). When the reaction was carried out without addition of hydrochloric acid, a large decrease in the catalyst activity during the reaction resulted. An increase in the hydrochloric acid content led to increased formation of the hydrogenolysis products relative to tetrahydrofurfuryl alcohol. [20, 21]

Smith and Fuzek used acetic acid as solvent and achieved virtually quantitative conversion into 1,2-pentanediol at a hydrogen pressure of 1.4-4 bar. [22]

In contrast, the reaction of furfural over supported platinum catalysts has likewise been described. Furfural was reacted at 110-150° C. and hydrogen pressures of from 15 to 25 bar over supported platinum catalysts in 2011 by Xu et al. [23] Pt/Co₂AlO₄ and Pt/Co₃O₄ catalysts prepared by coprecipitation and a Pt/Al₂O₃ catalyst produced by impregnation, in each case having a platinum content of 1.9%, were studied at 130° C. and a hydrogen pressure of 15 bar. After a reaction time of 24 hours, 2-methylfuran (2-MF) 1,2-methyltetrahydrofuran (2), 1-pentanol (3), 2-pentanol (4), tetrahydrofurfuryl alcohol (5), 1,2-pentanediol (6), 1,4-pentanediol (1,4-PD) (7) and 1,5-pentanediol (8) occurred as products. The yields are summarized in Table 1. The highest yield of 1,2-pentanediol, viz. 14.8%, was achieved using Pt/Co₃O₄. Pt/Co₂AlO₄ displayed an extraordinarily high catalytic activity compared to Pt/Al₂O₃. According to Xu et al., the high dispersity of platinum on the mesoporous cobalt aluminate support and the strong adsorption of the C—C double bond on Co(III) are responsible for this. Furfural was in each case rapidly hydrogenated to furfuryl alcohol, which reacted virtually completely to form the products 1 to 8. It was able to be shown that the formation of 1,2- and 1,5-pentanediol results from the hydrogenolysis of the furan ring of furfuryl alcohol and does not proceed via tetrahydrofurfuryl alcohol as intermediate. The hydrogenolysis of tetrahydrofurfuryl alcohol over platinum catalysts was found to be extremely difficult and led only to small conversions. Xu et al. attributed the formation of 1,4-pentanediol 7 to isomerization of 1,2-pentanediol. This played a role especially at high reaction temperatures and led to a reduction in the yield of 1,2-pentanediol (Table 2). The reaction paths which according to Xu et al. lead to the various reaction products are shown in Scheme 1. Here and in the following, the following abbreviations are used:

### Substances

1,2-BD: 1,2-Butanediol; 1,2-PD: 1,2-pentanediol; 1,4-PD: 1,4-pentanediol; 1,5-PD: 1,5-pentanediol; 1,5-HD: 1,5-hexanediol; 1-BOH: 1-butanol; 1-POH: 1-pentanol; 2-POH: 2-pentanol; 2-MF: 2-methylfuran; 2-MTHF: 2-methyltetrahydrofuran; CpO: cyclopentanone; CpOH: cyclopentanol; DCA: dicyanamide; IL: ionic liquid; PANI: polyaniline; FFOH: furfuryl alcohol; THF: tetrahydrofuran; THFFOH: tetrahydrofurfuryl alcohol.

### Physical Parameters

c: Concentration by mass; m: mass; n: number of moles; p: pressure; T: temperature; t: time; V: volume.

Physical units

° C.: Degrees Celsius; microlitre; micron; bar: bar; cm: centimetre; g: gram; h: hour; 1: litre; m: metre; mg: milligram; ml: millilitre; mol: mol; mmol: mmol; N: normality [mol/l]; min: minute.

### Further Abbreviations

Σ: Balance [%]; FID: flame ionization detector; GC: gas chromatography; GC-MS: gas chromatography coupled with mass spectrometry; K: calibration factor [mmol/(1 peak area)]; Cat: catalyst; Me: second metal; rpm: revolutions per minute; S: selectivity [%]; SCILL: solid catalyst with ionic liquid layer; SILP: supported ionic liquid phase; X: conversion [%]; Y: yield [%]; AC: activated carbon; AlOₓ: aluminum oxide.

TABLE 1
Yields in the reaction of furfural over supported Pt catalysts according
to Xu et al. [23]. Y(1-4) corresponds to the sum of the yields of 2-MF,
2-MTHF, 1-POH and 2-POH. Reaction conditions: T = 130° C.,
p(H₂) = 15 bar, t = 24 h.

| | Y(1-4) | Y(THFFOH) | Y(1,2-PD) | Y(1,4-PD) | Y(1,5-PD) |
|---|---|---|---|---|---|
| Catalyst | [%] | [%] | [%] | [%] | [%] |
| Pt/Co₂AlO₄ | 32.6 | 31.1 | 7.1 | 4.5 | 24.6 |
| Pt/Co₃O₄ | 20.1 | 21.8 | 14.8 | 2.8 | 30.8 |
| Pt/Al₂O₃ | 10.3 | 11.9 | 1.8 | 0.6 | 3.2 |

TABLE 2
Yields in the reaction of furfural over Pt/Co₂AlO₄ at different reaction
temperatures according to Xu et al. [23]. Y(1-4) corresponds to the sum
of the yields of 2-MF, 2-MTHF, 1-POH and 2-POH. Reaction conditions:
p(H₂) = 15 bar, t = 24 h.

| | Y(1-4) | Y(THFFOH) | Y(1,2-PD) | Y(1,4-PD) | Y(1,5-PD) |
|---|---|---|---|---|---|
| T [° C.] | [%] | [%] | [%] | [%] | [%] |
| 110 | 25.5 | 26.8 | 10.3 | 1.0 | 30.6 |
| 120 | 27.7 | 24.4 | 10.4 | 2.0 | 30.3 |
| 130 | 32.6 | 31.1 | 7.1 | 4.5 | 24.6 |

The reaction paths in the hydrogenolysis of furfural over platinum catalysts according to Xu et al. [11] are shown in Scheme 1 below. Here, 2-methylfuran (2-MF) is (1), 2-methyltetrahydrofuran is (2), 1-pentanol is (3), 2-pentanol is (4), tetrahydrofurfuryl alcohol is (5), 1,2 pentanediol is (6), 1,4-pentanediol (1,4-PD) is (7) and 1,5-pentanediol is (8).

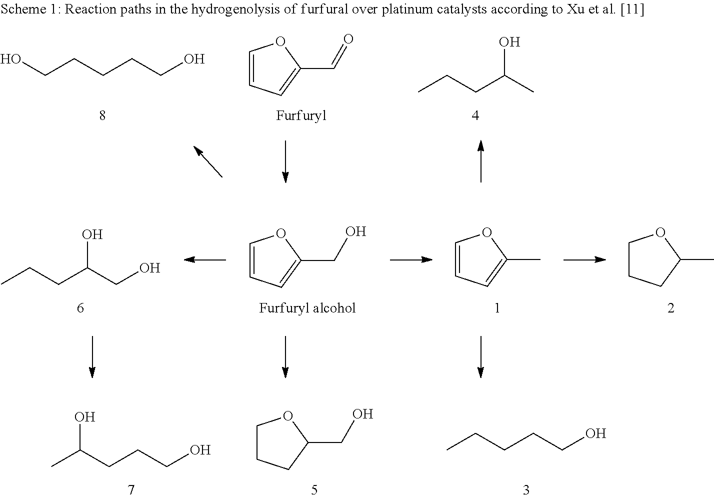

The hydrogenolysis of furfuryl alcohol over heterogeneous copper catalysts has also been described. As early as 1931, Adkins [24] reacted furfuryl alcohol over a copper chromite catalyst prepared by precipitation from basic solution of ammonium dichromate and copper nitrate. At a reaction temperature of 175° C. and a hydrogen pressure of 100-150 bar, 1,2-pentanediol 6 was formed as main product in a yield of 40%. In addition, 1,5-pentanediol 8 (Y=30%), pentanol (Y=10%) and also tetrahydrofurfuryl alcohol 5 and 2-methyltetrahydrofuran 2 occurred as by-products in unknown yields.

Manly [25] achieved a yield of up to 4.4% of 1,2-pentanediol 6 at a reaction temperature of 175° C. using copper chromite catalysts, with 2-methylfuran (1) being formed as main product in a yield of 66.4%. In addition, 2-methyltetrahydrofuran (2) and 2-pentanol (4) occurred as by-products. 1,5-Pentanediol (8) was not formed. The formation of 1,2-pentanediol (6) was attributed by Manly to the hydrogenolysis of furfuryl alcohol formed as intermediate by hydrogenation of furfural. Hydrogenolysis of tetrahydrofurfuryl alcohol to 1,2-pentanediol (5) did not take place under these reaction conditions. Based on these observations, Manly constructed the following reaction scheme, which encompasses all products detected.

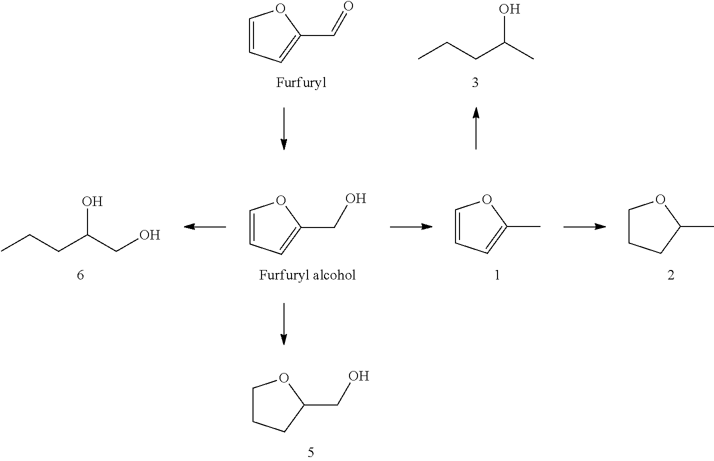

The use of copper oxides for the catalytic hydrogenolysis of furfuryl alcohol has also been described by Hachihama et al. [52]

Nickel catalysts, too, have been used to hydrogenate furfural and furfuryl alcohol with very high selectivity of up to 100% to tetrahydrofurfuryl alcohol. Catalysts used here were in particular supported nickel catalysts, alloy catalysts and Raney nickel. [20, 26, 27, 28] Typical reaction conditions are temperatures of 100-180° C. and hydrogen pressures in the range from 20 to 200 bar. According to Leuck et al., furfuryl alcohol can be converted in acidic aqueous solution over Raney nickel at 160° C. and 68 bar into a mixture of 1,2-pentanediol 6,1,4-pentanediol 7,1,5-pentanediol 8, 1,2,4-pentanetriol and tetrahydrofurfuryl alcohol, with the total proportion of diols in the product mixture being 40%. [29]

Nickel catalysts have likewise been described in the preparation of 1,2-alkanediols, for instance by Wang et al. [50] The use of nickel catalysts in the preparation of tetrahydrofurfuryl alcohol is described by Zhao et al. [51] The use of nickel-aluminum alloys for the hydrogenolysis of furan derivatives is described by Papa et al. in [53]. Copper-chromium-based catalysts are described by Connor & Adkins. [54]

Finally, the hydrogenation of furfural and furfuryl alcohol to tetrahydrofurfuryl alcohol over different ruthenium catalysts has been described. [26, 30, 31, 32] The reaction is generally carried out in methanol under mild reaction conditions. At a reaction temperature of 40° C. and a hydrogen pressure of 20 bar, a selectivity to tetrahydrofurfuryl alcohol of above 99% was achieved using ruthenium on a hectorite support. [30] When using RuO₂ at 120° C. and 50 bar, undefined, high molecular weight by-products were observed. [26] The use of ruthenium catalysts for the conversion of furfuryl alcohol into 1,2-pentanediol was also described by Zhang et al. [49]. Here, various ruthenium catalysts are used, but these are supported on support materials which are difficult to handle and are, in particular, unsuitable for industrial use. Zhang et al. accordingly describe a process which uses difficult-to-handle supports such as MnO₂ or AlMgO₄ which especially in industrial applications pose problems [49]. In addition, MnO₂ is unattractive compared to aluminum oxide, in particular Al₂O₃, as support material for industrial processes. Al₂O₃ is, especially due to its electric insulation properties, its mechanical strength, its high compressive strength, its high hardness, its moderate thermal conductivity, its high corrosion and wear resistance, its good sliding properties, its low density and its ability to be used even at high temperatures, a very attractive support material.

Another way of synthesizing 1,2-pentanediol is hydrogenolysis of tetrahydrofurfuryl alcohol which can be obtained from furfural or furfuryl alcohol by hydrogenation. [20, 26, 27, 28, 30, 31, 32]

Rhodium has been identified as the active metal of choice for hydrolysing tetrahydrofurfuryl alcohol under moderate reaction conditions. [58] Tomishige et al. were able to produce 1,5-pentanediol in a yield of up to 77% without 1,2-pentanediol being formed (Table 3) using Rh—ReOₓ/SiO₂ catalysts. [33] The reaction was carried out in aqueous solution at 120° C. and a hydrogen pressure of 80 bar. Under these reaction conditions, pure supported rhodium or rhenium catalysts gave only low conversions and preferentially formed 1,2-pentanediol. Thus, a selectivity of 61.7% to 1,2-pentanediol was achieved at a conversion of 5.7% when using Rh/SiO₂. By-products formed were mainly 1,5-pentanediol (S=18.0%) and 1-pentanol (S=6.2%). When using ReOₓ/SiO₂, the selectivity to 1,2-pentanediol was 31.2% and no 1,5-pentanediol was formed as by-product, with the conversion of tetrahydrofurfuryl alcohol being extremely low at less than 0.1%. Other active metals such as ruthenium, copper and nickel also display extremely low activity. According to Tomishige, the comparatively high activity of Rh—ReOₓ/SiO₂ and the high selectivity to 1,5-pentanediol are due to the formation of ReOₓ clusters on the surface of the Rh particles. The —CH₂OH group is adsorbed on these clusters, which makes it possible for the hydride ions formed at the Rh—ReOₓ interface to attack the adjacent C—O bond. [34]

TABLE 3
Conversions and selectivities in the reaction of THFFOH over
different catalysts according to Tomishige et al. [33].

| | | | | | | | S |
|---|---|---|---|---|---|---|---|
| | | | | S | S | S | (oth- |
| | T | t | X | (1,5-PD) | (1,2-PD) | (1-POH) | ers) |
| Catalyst | [° C.] | [h] | [%] | [%] | [%] | [%] | [%] |
| Rh- | 120 | 24 | 96.2 | 80.1 | 0.0 | 15.9 | 4.0 |
| ReOₓ/ | | | | | | | |
| SiO₂ | | | | | | | |
| Rh/SiO₂ | 120 | 4 | 5.7 | 18.0 | 61.7 | 6.2 | 14.1 |
| ReOx/ | 120 | 4 | <0.1 | 0.0 | 31.2 | 5.1 | 63.7 |
| SiO₂ | | | | | | | |
| Ru/C | 120 | 4 | 5.0 | 15.1 | 26.1 | 7.8 | 51.0 |
| Copper | 180 | 4 | 0.6 | 0.0 | 9.4 | 3.0 | 87.6 |
| chromite | | | | | | | |
| Raney Ni | 180 | 4 | 0.2 | 13.1 | 12.7 | 8.9 | 65.2 |

Reaction conditions: p (H₂) = 80 bar, 5% THFFOH in water.

Suzuki et al. hydrolysed tetrahydrofurfuryl alcohol over Rh and Pd catalysts under mild reaction conditions by using supercritical CO₂ as solvent. [35] When using rhodium on the mesoporous SiO₂ support MCM-41, tetrahydrofurfuryl alcohol was converted at 80° C. and a hydrogen pressure of 40 bar into 1,5-pentanediol with a selectivity of 91.2%, with 1-pentanol being formed as sole by-product. According to Suzuki et al., the presence of Rh₂O₃ particles in Rh/MCM-41 is responsible for the high activity and selectivity of the catalyst. When the catalyst was reduced at 300° C. so that only Rh(0) was present before the reaction, the activity decreased significantly and the selectivity changed in favour of 1,2-pentanediol (S=82.5%). Further Rh and Pd catalysts under the same reaction conditions gave product mixtures of 1,2-pentanediol, 1,5-pentanediol, 1-pentanol, 2-pentanol and other by-products (Table 4). The best yield of 1,2-pentanediol, viz. 39.1%, was given by Pd/MCM-41.

TABLE 4
Conversions and selectivities in the reaction of THFFOH over Rh and
Pd catalysts in supercritical CO₂ according to Suzuki et al. [35].
Reaction conditions: T = 80° C., p(H₂) = 40 bar,
^{a}catalyst was reduced at 300° C. before the reaction.

| | X | S (1,5-PD) | S (1,2-PD) | S (1-POH) | S (others) |
|---|---|---|---|---|---|
| Catalyst | [%] | [%] | [%] | [%] | [%] |
| Rh/MCM- | 80.5 | 91.2 | 0.0 | 8.8 | 0.0 |
| 41 | | | | | |
| Rh/MCM- | 5.0 | 12.1 | 82.5 | 5.4 | 0.0 |
| 41^{a} | | | | | |
| Rh/Al₂O₃ | 60.0 | 21.9 | 51.4 | 19.5 | 7.2 |
| Rh/C | 34.8 | 29.8 | 42.3 | 20.0 | 7.9 |
| Rh/SiO₂ | 30.4 | 78.2 | 11.4 | 8.2 | 12.2 |
| Pd/MCM- | 50.5 | 12.6 | 77.4 | 10.0 | 0.0 |
| 41 | | | | | |
| Pd/Al₂O₃ | 32.5 | 9.1 | 0.9 | 47.5 | 42.5 |
| Pd/C | 48.9 | 99.2 | 0.3 | 0.5 | 0.0 |

Reported results on the hydrogenolysis of furfuryl alcohol or tetrahydrofurfuryl alcohol to 1,2-pentanediol are summarized in Table 5. The best yields were achieved using Pt(IV) oxide in acetic acid as solvent, but no precise information regarding the reaction conditions is available.

TABLE 5
Summary of reported results for the hydrogenolysis of furfuryl
alcohol or THFFOH to 1,2-pentanediol.

| | Starting | | | p(H2) | Y(1,2-PD) |
|---|---|---|---|---|---|
| Catalyst | material | Solvent | T [° C.] | [bar] | [%] |
| Pt(IV) | Furfural | Ethanol | 25 | 1-2 | 20 |
| Oxide [18] | | | | | |
| Pt(IV) | FFOH | Ethanol | 25 | 1 | 13.3 |
| Oxide [20] | | | | | |
| Pt(IV) | FFOH | Acetic acid | Unknown | 1.4-4 | ca. 100 |
| Oxide [22] | | | | | |
| Pt/Co₃O₄ | Furfural | None | 130 | 15 | 14.8 |
| [23] | | | | | |
| Raney Ni | FFOH | Water | 160 | 68 | Unknown^{a)} |
| [29] | | | | | |
| Copper | FFOH | None | 175 | 100-150 | 40 |
| chromite | | | | | |
| [24] | | | | | |
| Rh/SiO₂ | THFFOH | Water | 120 | 80 | 3.5 |
| [33] | | | | | |
| Pd/MCM- | THFFOH | CO₂ | 80 | 40 | 39.1 |
| 41 [35] | | | | | |

^{a)}The total yield of 1,2-pentanediol, 1,4-pentanediol and 1,5-pentanediol is 40%.

The term ionic liquids (IL) generally refers to salts which have a melting point below 100° C. The first IL which was liquid at room temperature was described in 1914 and was ethylammonium nitrate. [36] Ionic liquids have promising properties which make them particularly interesting for green chemistry. They display, inter alia, a high stability, good solvent properties and a negligible vapour pressure. [37] Despite these advantages, research interest in their use as solvents or catalysts was awakened only in the 1980s. [38, 39] A particularly current field of research is immobilization of ionic liquids on solid surfaces. Here, a distinction is made between the two basic concepts of SILP (Supported Ionic Liquid Phase) and SCILL (Solid Catalyst with Ionic Liquid Layer). [40] SILP is a variant of homogeneous catalysis in which the catalyst is dissolved in an IL immobilized on a solid support. In contrast, SCILL describes coating of a heterogeneous catalyst with a thin IL film. This IL layer influences the course of the reaction by means of the altered solubilities of the reactants compared to the solvent. In addition, direct interactions between IL and catalyst can also occur, for example by electronic ligand effects similar to those of second metals [41, 42]. The inventors of the present invention were able to achieve selectivities of above 99% in the hydrogenation of citral to citronellal over Pd catalysts by coating the catalyst with small amounts of an ionic liquid. The best selectivities were given by ionic liquids containing the dicyanamide anion (DCA). [43, 44] It was shown that DCA coordinates to Pd and thereby weakens the adsorption of hydrogen, which prevents the further reaction of citronellal to form undesirable subsequent products. [42]

It was therefore an object of the present invention to provide a process for the reaction of furfuryl alcohol, which in comparison to conventionally known catalysts displays a high selectivity to 1,2-pentanediol and at the same time proceeds with a readily processable and widely available catalyst. The process should advantageously also lead to a high yield of 1,2-pentanediol, even to a very high total yield of tetrahydrofurfuryl alcohol. Such a process would be particularly advantageous since tetrahydrofurfuryl alcohol may also be used as solvent and is therefore an important product, in addition to 1,2-pentanediol. In addition, it is desirable for such a process to proceed to completion in order to ensure that the toxic starting material furfuryl alcohol is completely reacted.

### SUMMARY OF THE INVENTION

It has now surprisingly been found that this object and others may be achieved by the process of the present invention which includes a ruthenium catalyst supported on aluminum oxide, in particular Al₂O₃, or activated carbon (AC), or platinum(IV) oxide. The process of the invention surprisingly leads with high selectivity to the product 1,2-pentanediol and at the same time with high selectivity to tetrahydrofurfuryl alcohol. Furthermore, the process may achieve virtually complete conversion.

The present invention provides, in a first aspect, each of the following embodiments:

1.1 Process for preparing 1,2-pentanediol, characterized in that furfuryl alcohol is reacted with hydrogen in the presence of an Ru/AlOₓ catalyst system.

For the purposes of the invention, the expression “Ru/AlOₓ catalyst system” is used as an abbreviation for “a catalyst system comprising (I) ruthenium and (II) a support composed of aluminum oxide on which the ruthenium is supported”.

1.2. Process according to embodiment 1.1, wherein the aluminum oxide is Al₂O₃.
1.3. Process according to embodiment 1.1 or 1.2, wherein the content of ruthenium is from 0.01 to 30% by weight, based on the total weight of Ru/AlOₓ catalyst system.
1.4. Process according to one or more of embodiments 1.1 to 1.3, wherein the Ru/AlOₓ catalyst system has a BET surface area of from 50 to 250 m²/g of Ru/AlOₓ catalyst system.
1.5. Process according to one or more of embodiments 1.1 to 1.4, wherein the Ru/AlOₓ catalyst system has an average pore volume of from 0.2 to 0.8 ml/g of Ru/AlOₓ catalyst system.
1.6. Process according to one or more of embodiments 1.1 to 1.5, wherein the Ru/AlOₓ catalyst system has the following X-ray diffraction pattern:

| | Relative |
|---|---|
| | intensity |
| Pos. [°2Th.] | [%] |
| 18.3 | 5-10 |
| 18.8 | 12-17 |
| 20.33 | 6-11 |
| 31.37 | 47-53 |
| 32.68 | 25-32 |
| 32.825 | 80-85 |
| 35.09 | 10-15 |
| 36.758 | 53-59 |
| 38.97 | 27-32 |
| 39.8 | 30-35 |
| 40.63 | 8-13 |
| 43.359 | 14-18 |
| 44.85 | 62-67 |
| 45.47 | 45-50 |
| 46.54 | 25-30 |
| 47.61 | 30-35 |
| 50.77 | 8-13 |
| 51.5 | 7-12 |
| 57.48 | 7-11 |
| 59.97 | 15-20 |
| 62.38 | 8-13 |
| 62.848 | 8-13 |
| 64.05 | 10-16 |
| 65.7 | 10-15 |
| 66.52 | 54-59 |
| 67 | 31-37 |
| 67.4 | 100 |
| 73.5 | 5-10 |

1.7. Process according to one or more of embodiments 1.1 to 1.6, wherein it is carried out at a temperature of 100° C.-280° C.
1.8. Process according to one or more of embodiments 1.1 to 1.7, wherein it is carried out under autoclave conditions. Here, “autoclave conditions” means, for the purposes of the invention, a pressure of equal to or greater than 10 bar.
1.9. Process according to one or more of embodiments 1.1 to 1.8, wherein the process is carried out in a solvent selected from the group consisting of water, ethanol, tetrahydrofuran and 1,4-dioxane.
1.10. Process according to one or more of embodiments 1.1 to 1.9, wherein it is carried out at a pH of from 5.3 to 10.5.
1.11. Process according to one or more of embodiments 1.1 to 1.10, wherein it is carried out continuously.
1.12. Process according to one or more of embodiments 1.1 to 1.11, wherein it is carried out batchwise.
1.13. Process according to one or more of embodiments 1.1 to 1.12, wherein the Ru/AlOₓ catalyst system is obtained by a process comprising:

a) Producing a mixture (i) comprising a ruthenium salt solution (ii) and an aqueous suspension of aluminum oxide (iii);

b) Bringing the mixture (i) to a temperature in the range from greater than 0° C. to less than 100° C. and a pH of 0-14;

c) Separating off the Ru/AlOₓ catalyst system by filtering the mixture (i);

d) Drying the Ru/AlOₓ catalyst system separated off in step c).

1.14. Process for producing an Ru/AlOₓ catalyst system, which comprises

a) Producing a mixture (i) comprising a ruthenium salt solution (ii) and an aqueous suspension of aluminum oxide (iii);

b) Bringing the mixture (i) to a temperature in the range from greater than 0° C. to less than 100° C. and a pH of 0-14;

c) Separating off the Ru/AlOₓ catalyst system by filtering the mixture (i);

d) Drying the Ru/AlOₓ catalyst system separated off in c).

1.15 Process according to embodiment 1.14, wherein a reducing agent (iv) is added to the mixture (i) before b) or between b) and c), preferably between steps b) and c).
1.16 Process according to embodiment 1.14 or 1.15, wherein a temperature in the range 55-75° C. and a pH of 7.5-8.5 are set in step b) by addition of a base B2 to the mixture (i).
1.17. Ru/AlOₓ Catalyst system obtained by a process according to one or more of embodiments 1.14 to 1.16.

In a second aspect, the present invention provides the following embodiments:

2.1. Process for preparing 1,2-pentanediol, wherein furfuryl alcohol is reacted with hydrogen in the presence of an Ru/AC catalyst system.

For the purposes of the invention, the expression “Ru/AC catalyst system” is used as an abbreviation for “a catalyst system comprising (I) ruthenium and (II) a support composed of activated carbon on which the ruthenium is supported”.

2.2. Process according to embodiment 2.1, wherein the content of ruthenium is from 0.01 to 30% by weight, based on the total weight of the Ru/AC catalyst system.
2.3. Process according to embodiment 2.1 or 2.2, wherein the catalyst system has a BET surface area of from 300 to 2000 m²/g of Ru/AC catalyst system.
2.4. Process according to one or more of embodiments 2.1 to 2.3, wherein the Ru/AC catalyst system has an average pore volume of from 0.3 to 2.0 ml/g of Ru/AC catalyst system.
2.5. Process according to one or more of embodiments 2.1 to 2.4, wherein it is carried out at a temperature of 100° C.-280° C.
2.6. Process according to one or more of embodiments 2.1 to 2.5, wherein it is carried out under autoclave conditions. Here, the term “autoclave conditions” means, for the purposes of the invention, a pressure of equal to or greater than 10 bar.
2.7. Process according to one or more of embodiments 2.1 to 2.6, wherein the process is carried out in a solvent selected from the group consisting of water, ethanol, tetrahydrofuran and 1,4-dioxane.
2.8. Process according to one or more of embodiments 2.1 to 2.7, wherein it is carried out at a pH of from 5.3 to 10.5.
2.9. Process according to one or more of embodiments 2.1 to 2.8, wherein it is carried out continuously.
2.10. Process according to one or more of embodiments 2.1 to 2.9, wherein it is carried out batchwise.
2.11. Process according to one or more of embodiments 2.1 to 2.10, wherein the Ru/AC catalyst system is obtained as follows:

a) Producing a mixture (i) comprising a ruthenium salt solution (ii) and an aqueous suspension of activated carbon (iii);

b) Bringing the mixture (i) to a temperature in the range from greater than 0° C. to less than 100° C. and a pH of 0-14;

c) Separating off the Ru/AC catalyst system by filtering the mixture (i).

2.12. Process for producing an Ru/AC catalyst system, which comprises

a) Producing a mixture (i) comprising a ruthenium salt solution (ii) and an aqueous suspension of activated carbon (iii);

b) Bringing the mixture (i) to a temperature in the range from greater than 0° C. to less than 100° C. and a pH of 0-14;

c) Separating off the Ru/AC catalyst system by filtering the mixture (i).

2.13. Process according to embodiment 2.12, wherein a reducing agent (iv) is added to the mixture (i) before b) or between b) and c), preferably between steps b) and c).
2.14. Process according to embodiment 2.12 or 2.13, wherein a temperature in the range 55-75° C. and a pH of 7.5-8.5 is set in b) by addition of a base B2 to the mixture (i).
2.15. Ru/AlOₓ catalyst system obtained by a process according to one or more of embodiments 2.12 to 2.14.

In a third aspect, the present invention provides the following embodiments:

3.1. Process for preparing 1,2-pentanediol, wherein furfuryl alcohol is reacted with hydrogen in the presence of Pt(IV) oxide.
3.2. Process according to embodiment 3.1, wherein it is carried out at a temperature greater than 0° C. and less than 100° C.
3.3. Process according to embodiment 3.1 or 3.2, wherein it is carried out at a pressure of 1-10 bar.
3.4. Process according to one or more of embodiments 3.1 to 3.3, wherein the process is carried out in a solvent selected from the group consisting of water, acetic acid, ethanol.
3.5. Process according to one or more of embodiments 3.1 to 3.4, wherein the process is carried out with addition of hydrochloric acid.

In a fourth aspect, the present invention provides the following embodiments:

4.1. Process for preparing tetrahydrofurfuryl alcohol, wherein furfuryl alcohol is reacted with hydrogen in the presence of an Ru/AlOₓ catalyst system.
4.2. Process according to embodiment 4.1, wherein the aluminum oxide is Al₂O₃.
4.3. Process according to embodiments 4.1 or 4.2, wherein the content of ruthenium is from 0.01 to 30% by weight, based on the total weight of the Ru/AlOₓ catalyst system.
4.4. Process according to one or more of embodiments 4.1 to 4.3, wherein the Ru/AlOₓ catalyst system has a BET surface area of from 50 to 250 m²/g of Ru/AlOₓ catalyst system.
4.5. Process according to one or more of embodiments 4.1 to 4.4, wherein the Ru/AlOₓ catalyst system has an average pore volume of from 0.2 to 0.8 ml/g of Ru/AlOₓ catalyst system.
4.6. Process according to one or more of embodiments 4.1 to 4.5, wherein the Ru/AlOₓ catalyst system has the following X-ray diffraction pattern:

| | Relative |
|---|---|
| | intensity |
| Pos. [°2Th.] | [%] |
| 18.3 | 5-10 |
| 18.8 | 12-17 |
| 20.33 | 6-11 |
| 31.37 | 47-53 |
| 32.68 | 25-32 |
| 32.825 | 80-85 |
| 35.09 | 10-15 |
| 36.758 | 53-59 |
| 38.97 | 27-32 |
| 39.8 | 30-35 |
| 40.63 | 8-13 |
| 43.359 | 14-18 |
| 44.85 | 62-67 |
| 45.47 | 45-50 |
| 46.54 | 25-30 |
| 47.61 | 30-35 |
| 50.77 | 8-13 |
| 51.5 | 7-12 |
| 57.48 | 7-11 |
| 59.97 | 15-20 |
| 62.38 | 8-13 |
| 62.848 | 8-13 |
| 64.05 | 10-16 |
| 65.7 | 10-15 |
| 66.52 | 54-59 |
| 67 | 31-37 |
| 67.4 | 100 |
| 73.5 | 5-10 |

4.7. Process according to one or more of embodiments 4.1 to 4.6, wherein the hydrogenolysis is conducted at a temperature of 100° C.-280° C.
4.8. Process according to one or more of embodiments 4.1 to 4.7, wherein the hydrogenolysis is conducted under autoclave conditions. Here, “autoclave conditions” means, for the purposes of the invention, a pressure of equal to or greater than 10 bar.
4.9. Process according to one or more of embodiments 4.1 to 4.8, wherein the hydrogenolysis is conducted in a solvent selected from the group consisting of water, ethanol, tetrahydrofuran and 1,4-dioxane.
4.10. Process according to one or more of embodiments 4.1 to 4.9, wherein the hydrogenolysis is conducted at a pH of from 5.3 to 10.5.
4.11. Process according to one or more of embodiments 4.1 to 4.10, wherein the hydrogenolysis is conducted continuously.
4.12. Process according to one or more of embodiments 4.1 to 4.11, wherein the hydrogenolysis is conducted batchwise.
4.13. Process according to one or more of embodiments 4.1 to 4.12, wherein the Ru/AlOₓ catalyst system is obtained by a method comprising:

a) producing a mixture (i) comprising a ruthenium salt solution (ii) and an aqueous suspension of aluminum oxide (iii);

b) bringing the mixture (i) to a temperature in the range from greater than 0° C. to less than 100° C. and a pH of 0-14;

c) separating off the Ru/AlOₓ catalyst system by filtering the mixture (i);

d) drying the Ru/AlOₓ catalyst system separated off in c).

In a fifth aspect the present invention provides the following embodiments:

5.1. Process for preparing tetrahydrofurfuryl alcohol, wherein furfuryl alcohol is reacted with hydrogen in the presence of an Ru/AC catalyst system.
5.2 Process according to embodiment 5.1, wherein the content of ruthenium is from 0.01 to 30% by weight, based on the total weight of the Ru/AC catalyst system.
5.3. Process according to embodiment 5.1 or 5.2, wherein the catalyst system has a BET surface area of from 300 to 2000 m²/g of Ru/AC catalyst system.
5.4. Process according to one or more of embodiments 5.1 to 5.3, wherein the Ru/AC catalyst system has an average pore volume of from 0.3 to 2.0 ml/g of Ru/AC catalyst system.
5.5. Process according to one or more of embodiments 5.1 to 5.4, wherein the hydrogenolysis is conducted at a temperature of 100° C.-280° C.
5.6. Process according to one or more of embodiments 5.1 to 5.5, wherein the hydrogenolysis is conducted under autoclave conditions. Here, the term “autoclave conditions” means, for the purposes of the invention, a pressure of equal to or greater than 10 bar.
5.7. Process according to one or more of embodiments 5.1 to 5.6, wherein the hydrogenolysis is conducted in a solvent selected from the group consisting of water, ethanol, tetrahydrofuran and 1,4-dioxane.
5.8. Process according to one or more of embodiments 5.1 to 5.7, wherein the hydrogenolysis is conducted at a pH of from 5.3 to 10.5.
5.9. Process according to one or more of embodiments 5.1 to 5.8, wherein the hydrogenolysis is conducted continuously.
5.10. Process according to one or more of embodiments 5.1 to 5.9, wherein the hydrogenolysis is conducted batchwise.
5.11. Process according to one or more of embodiments 5.1 to 5.10, wherein the Ru/AC catalyst system is obtained by a method comprising:

a) producing a mixture (i) comprising a ruthenium salt solution (ii) and an aqueous suspension of activated carbon (iii);

b) bringing the mixture (i) to a temperature in the range from greater than 0° C. to less than 100° C. and a pH of 0-14;

c) separating off the Ru/AC catalyst system by filtering the mixture (i).

In a sixth aspect the present invention provides the following embodiments:

6.1. Process for preparing tetrahydrofurfuryl alcohol, wherein furfuryl alcohol is reacted with hydrogen in the presence of Pt(IV) oxide.
6.2. Process according to embodiment 6.1, wherein the hydrogenolysis is conducted at a temperature of greater than 0° C. and less than 100° C.
6.3. Process according to embodiment 6.1 or 6.2, wherein the hydrogenolysis is conducted at a pressure of 1-10 bar.
6.4. Process according to one or more of embodiments 6.1 to 6.3, wherein the hydrogenolysis is conducted in a solvent selected from the group consisting of water, acetic acid, and ethanol.
6.5. Process according to one or more of embodiments 6.1 to 6.4, wherein the hydrogenolysis is conducted with addition of hydrochloric acid.

In a seventh aspect the present invention provides the following embodiments:

7.1. Process for preparing 1,2-pentanediol and tetrahydrofurfuryl alcohol, wherein furfuryl alcohol is reacted with hydrogen in the presence of an Ru/AlOₓ catalyst system.
7.2. Process according to embodiment 7.1, wherein the aluminum oxide is Al₂O₃.
7.3. Process according to embodiment 7.1 or 7.2, wherein the content of ruthenium is from 0.01 to 30% by weight, based on the total weight of Ru/AlOₓ catalyst system.
7.4. Process according to one or more of embodiments 7.1 to 7.3, wherein the Ru/AlOₓ catalyst system has a BET surface area of from 50 to 250 m²/g of Ru/AlOₓ catalyst system.
7.5. Process according to one or more of embodiments 7.1 to 7.4, wherein the Ru/AlOₓ catalyst system has an average pore volume of from 0.2 to 0.8 ml/g of Ru/AlOₓ catalyst system.
7.6. Process according to one or more of embodiments 7.1 to 7.5, wherein the Ru/AlOₓ catalyst system has the following X-ray diffraction pattern:

| | Relative |
|---|---|
| | intensity |
| Pos. [°2Th.] | [%] |
| 18.3 | 5-10 |
| 18.8 | 12-17 |
| 20.33 | 6-11 |
| 31.37 | 47-53 |
| 32.68 | 25-32 |
| 32.825 | 80-85 |
| 35.09 | 10-15 |
| 36.758 | 53-59 |
| 38.97 | 27-32 |
| 39.8 | 30-35 |
| 40.63 | 8-13 |
| 43.359 | 14-18 |
| 44.85 | 62-67 |
| 45.47 | 45-50 |
| 46.54 | 25-30 |
| 47.61 | 30-35 |
| 50.77 | 8-13 |
| 51.5 | 7-12 |
| 57.48 | 7-11 |
| 59.97 | 15-20 |
| 62.38 | 8-13 |
| 62.848 | 8-13 |
| 64.05 | 10-16 |
| 65.7 | 10-15 |
| 66.52 | 54-59 |
| 67 | 31-37 |
| 67.4 | 100 |
| 73.5 | 5-10 |

7.7. Process according to one or more of embodiments 7.1 to 7.6, wherein the hydrogenolysis is conducted at a temperature of 100° C.-280° C.
7.8. Process according to one or more of embodiments 7.1 to 7.7, wherein the hydrogenolysis is conducted under autoclave conditions. Here, “autoclave conditions” means, for the purposes of the invention, a pressure of equal to or greater than 10 bar.
7.9. Process according to one or more of embodiments 7.1 to 7.8, wherein the hydrogenolysis is conducted in a solvent selected from the group consisting of water, ethanol, tetrahydrofuran and 1,4-dioxane.
7.10. Process according to one or more of embodiments 7.1 to 7.9, wherein the hydrogenolysis is conducted at a pH of from 5.3 to 10.5.
7.11. Process according to one or more of embodiments 7.1 to 7.10, wherein the hydrogenolysis is conducted continuously.
7.12. Process according to one or more of embodiments 7.1 to 7.11, wherein the hydrogenolysis is conducted batchwise.
7.13. Process according to one or more of embodiments 7.1 to 7.12, wherein the Ru/AlOₓ catalyst system is obtained by a method comprising:

a) producing a mixture (i) comprising a ruthenium salt solution (ii) and an aqueous suspension of aluminum oxide (iii);

b) bringing the mixture (i) to a temperature in the range from greater than 0° C. to less than 100° C. and a pH of 0-14;

c) separating off the Ru/AlOₓ catalyst system by filtering the mixture (i);

d) drying the Ru/AlOₓ catalyst system separated off in c).

In an eighth aspect the present invention provides the following embodiments:

8.1. Process for preparing 1,2-pentanediol and tetrahydrofurfuryl alcohol, wherein furfuryl alcohol is reacted with hydrogen in the presence of an Ru/AC catalyst system.
8.2 Process according to embodiment 8.1, wherein the content of ruthenium is from 0.01 to 30% by weight, based on the total weight of the Ru/AC catalyst system.
8.3. Process according to embodiment 8.1 or 8.2, wherein the catalyst system has a BET surface area of from 300 to 2000 m²/g of Ru/AC catalyst system.
8.4. Process according to one or more of embodiments 8.1 to 8.3, wherein the Ru/AC catalyst system has an average pore volume of from 0.3 to 2.0 ml/g of Ru/AC catalyst system.
8.5. Process according to one or more of embodiments 8.1 to 8.4, wherein the hydrogenolysis is conducted at a temperature of 100° C.-280° C.
8.6. Process according to one or more of embodiments 8.1 to 8.5, wherein the hydrogenolysis is conducted under autoclave conditions. Here, the term “autoclave conditions” means, for the purposes of the invention, a pressure of equal to or greater than 10 bar.
8.7. Process according to one or more of embodiments 8.1 to 8.6, wherein the hydrogenolysis is conducted in a solvent selected from the group consisting of water, ethanol, tetrahydrofuran and 1,4-dioxane.
8.8. Process according to one or more of embodiments 8.1 to 8.7, wherein the hydrogenolysis is conducted at a pH of from 5.3 to 10.5.
8.9. Process according to one or more of embodiments 8.1 to 8.8, wherein the hydrogenolysis is conducted continuously.
8.10. Process according to one or more of embodiments 8.1 to 8.9, wherein the hydrogenolysis is conducted batchwise.
8.11. Process according to one or more of embodiments 8.1 to 8.10, wherein the Ru/AC catalyst system is obtained by a method comprising:

a) producing a mixture (i) comprising a ruthenium salt solution (ii) and an aqueous suspension of activated carbon (iii);

b) bringing the mixture (i) to a temperature in the range from greater than 0° C. to less than 100° C. and a pH of 0-14;

c) separating off the Ru/AC catalyst system by filtering the mixture (i).

In a ninth aspect the present invention provides the following embodiments:

9.1. Process for preparing 1,2-pentanediol and tetrahydrofurfuryl alcohol, wherein furfuryl alcohol is reacted with hydrogen in the presence of Pt(IV) oxide.
9.2. Process according to point 9.1, wherein the hydrogenolysis is conducted at a temperature of greater than 0° C. and less than 100° C.
9.3. Process according to embodiment 9.1 or 9.2, wherein the hydrogenolysis is conducted at a pressure of 1-10 bar.
9.4. Process according to one or more of embodiments 9.1 to 9.3, wherein the hydrogenolysis is conducted in a solvent selected from the group consisting of water, acetic acid, and ethanol.
9.5. Process according to one or more of embodiments 9.1 to 9.4, wherein the hydrogenolysis is conducted with addition of hydrochloric acid.

### DETAILED DESCRIPTION OF THE INVENTION

Throughout this description all ranges described include all values and sub-ranges therein, unless otherwise specified. Additionally, the indefinite article “a” or “an” carries the meaning of “one or more” throughout the description, unless otherwise specified.

The present invention provides, in a first aspect, a process for preparing 1,2-pentanediol, wherein furfuryl alcohol is reacted with hydrogen in the presence of a catalyst system comprising: (I) ruthenium and (II) a support of aluminum oxide.

For the purposes of the invention, the expression “Ru/AlOₓ catalyst system” is used as an abbreviation for “a catalyst system comprising (I) ruthenium and (II) a support composed of aluminum oxide on which the ruthenium is supported”.

An advantage of the present process according to the invention for preparing 1,2-pentanediol according to the first aspect is that aluminum oxide is used as support. This makes it possible to produce and use the catalyst industrially since the aluminum oxide support, in particular Al₂O₃, may readily be produced from the corresponding precursor compounds, for example from bayerite and/or pseudoboehmite. In addition, aluminum oxide is readily available and displays a good industrial processability and usability. These advantages which are ensured by aluminum oxides, in particular Al₂O₃, are not ensured by other conventional support materials exemplified by manganese oxides or spinels such as MgAlO₄ or MgAl₂O₄.

For the purposes of the present invention, “aluminum oxide” is, in particular, Al₂O₃. Al₂O₃ may be used in any modification. Al₂O₃ may preferably be used in a modification selected from among theta, gamma and mixtures thereof. For the purposes of the present invention, “aluminum oxide” is particularly preferably gamma-Al₂O₃. The preparation of the aluminum oxide according to the invention is conventionally known. The aluminum oxide may, for example, be prepared from aluminum oxide precursor compounds such as gibbsite [Al(OH)₃], bayerite [Al(OH)₃], boehmite [AlO(OH)] and/or pseudoboehmite. These aluminum oxide precursors may be treated with an acid, for example nitric acid, and subsequently shaped. The shaped bodies obtained in this way can then be dried (this can, for example, be carried out at temperatures of from 80 to 140° C.) and subsequently calcined at temperatures of from 400 to 700° C.

The content of ruthenium in the Ru/AlOₓ catalyst system may be in the range 0.01-30% by weight, preferably in the range 0.01-20% by weight, particularly preferably in the range 0.5-10.5% by weight, very particularly preferably in the range 5-10% by weight, most preferably 5% by weight, based on the total weight of the Ru/AlOₓ catalyst system. The values are measured after drying the supported catalyst at 80° C. under reduced pressure to constant weight. The content of ruthenium based on the total weight of the Ru/AlOₓ catalyst system may be determined in accordance with DIN51009.

The Ru/AlOₓ catalyst system may preferably be mesoporous and having a pore diameter in the range from 2 nm to 50 nm, determined in accordance with DIN ISO 9277. The total BET surface area of the Ru/AlOₓ catalyst system may be in the range from 50 to 250 m²/g of Ru/AlOₓ catalyst system, in particular from 100 to 150 m²/g of Ru/AlOₓ catalyst system. The total BET surface area is determined in accordance with DIN ISO 9277.

The pore volume of the Ru/AlOₓ catalyst system may be from 0.2 to 0.8 ml/g of Ru/AlOₓ catalyst system, preferably from 0.3 to 0.6 ml/g of Ru/AlOₓ catalyst system, particularly preferably 0.4 ml/g of Ru/AlOₓ catalyst system. The pore volume is determined in accordance with DIN ISO 9277.

The Ru/AlOₓ catalyst system preferably has the following X-ray diffraction pattern.

| Pos. | Relative |
|---|---|
| [°2Th.] | Intensity [%] |
| 18.3 | 5-10 |
| 18.8 | 12-17 |
| 20.33 | 6-11 |
| 31.37 | 47-53 |
| 32.68 | 25-32 |
| 32.825 | 80-85 |
| 35.09 | 10-15 |
| 36.758 | 53-59 |
| 38.97 | 27-32 |
| 39.8 | 30-35 |
| 40.63 | 8-13 |
| 43.359 | 14-18 |
| 44.85 | 62-67 |
| 45.47 | 45-50 |
| 46.54 | 25-30 |
| 47.61 | 30-35 |
| 50.77 | 8-13 |
| 51.5 | 7-12 |
| 57.48 | 7-11 |
| 59.97 | 15-20 |
| 62.38 | 8-13 |
| 62.848 | 8-13 |
| 64.05 | 10-16 |
| 65.7 | 10-15 |
| 66.52 | 54-59 |
| 67 | 31-37 |
| 67.4 | 100 |
| 73.5 | 5-10 |

The Ru/AlOₓ catalyst system more preferably has the following X-ray diffraction pattern.

| Pos. | Relative |
|---|---|
| [°2Th.] | Intensity [%] |
| 18.3 | 8.69 |
| 18.8 | 13.92 |
| 20.33 | 8.8 |
| 31.37 | 51.95 |
| 32.68 | 28.16 |
| 32.825 | 82.43 |
| 35.09 | 12.34 |
| 36.758 | 56.2 |
| 38.97 | 30.89 |
| 39.8 | 32.12 |
| 40.63 | 10.43 |
| 43.359 | 16.21 |
| 44.85 | 64.42 |
| 45.47 | 47.23 |
| 46.54 | 28.33 |
| 47.61 | 32.26 |
| 50.77 | 11.05 |
| 51.5 | 9.98 |
| 57.48 | 8.68 |
| 59.97 | 17.46 |
| 62.38 | 11.05 |
| 62.848 | 10.72 |
| 64.05 | 13.91 |
| 65.7 | 12.04 |
| 66.52 | 56.88 |
| 67 | 34.05 |
| 67.4 | 100 |
| 73.5 | 6.88 |

The particle size distribution of the Ru/AlOₓ catalyst system may typically be d10=2-8 μm; d50=20-40 μm; d90=50-80 μm. Here, “d10=2-8 μm” means that 10% of the Ru/AlOₓ catalyst system has a particle size of 2-8 μm or less; “d50=20-40 μm” means that 50% of the Ru/AlOₓ catalyst system has a particle size of 20-40 μm or less; and “d90=50-80 μm” means that 90% of the Ru/AlOₓ catalyst system has a particle size of 50-80 μm or less.

In the process of the invention for preparing 1,2-pentanediol according to the first aspect of the invention, the temperature is in principle not subject to any restrictions; thus, for example, it may be possible to set a temperature of greater than or equal to 100° C. The process of the invention for preparing 1,2-pentanediol according to the first aspect of the invention may preferably be conducted at a temperature of from 100° C. to 280° C., particularly preferably at a temperature of from 150 to 260° C. It has been found to be very particularly advantageous, and it may be therefore very particularly preferred, for the process of the invention for preparing 1,2-pentanediol according to the first aspect of the invention to be carried out at a temperature of from 200° C. to 240° C.

The process of the invention for preparing 1,2-pentanediol according to the first aspect of the invention may preferably be conducted under autoclave conditions. For the purposes of the invention, “autoclave conditions” describe pressures of equal to or greater than 10 bar, preferably pressures in the range from 10 to 200 bar, particularly preferably pressures in the range from 50 to 150 bar, most preferably a pressure of 100 bar.

The process of the invention for preparing 1,2-pentanediol according to the first aspect of the present invention may be conducted in any desired solvent. In particular, use is made in this context of solvents selected from the group consisting of water, ethanol, tetrahydrofuran, 1,4-dioxane. Particular preference may be given to using water as solvent.

The process of the invention according to the first aspect of the present invention may be conducted batchwise or continuously.

In the process of the invention for preparing 1,2-pentanediol according to the first aspect of the present invention, any gas which comprises free hydrogen and is free of additives which could suppress the process, for example carbon monoxide, may be used as hydrogen source.

The amount of Ru/AlOₓ catalyst system used in the process of the invention for preparing 1,2-pentanediol according to the first aspect of the present invention is not subject to any particular restrictions and may be in the range 0.01-30% by weight, preferably 0.5-30% by weight, particularly preferably 1-20% by weight, very particularly preferably 1-10% by weight, in each case based on the weight of the furfuryl alcohol used.

The Ru/AlOₓ catalyst system may comprise further metals selected from the group consisting of zinc, nickel, platinum and iron, preferably zinc and iron, particularly preferably iron, in addition to ruthenium. However, preference may be given to the Ru/AlOₓ catalyst system not containing any further metals in addition to ruthenium. For the purposes of the invention, “no further metal” means that the proportion of any further metals supported on the support composed of aluminum oxide in addition to ruthenium is, calculated on the basis of the dry weight of catalyst system, in the range from 0% by weight to 5% by weight, preferably from 0% by weight to 2.5% by weight, more preferably from 0% by weight to 1% by weight, particularly preferably from 0% by weight to 0.1% by weight, very particularly preferably from 0% by weight to 0.01% by weight, most preferably from 0% by weight to 0.001% by weight (able to be determined by means of the method of DIN51009).

In addition, it has surprisingly been found that particularly good selectivities to 1,2-pentanediol may be obtained when the process of the invention for preparing 1,2-pentanediol according to the first aspect of the present invention is conducted at a pH of from 5.3 to 10.3, more preferably from 6.2 to 10.2, most preferably at a pH of 7.6. This may, for example, be achieved by adding a base, for example Na₂CO₃, before the reaction. The amount of base necessary for achieving the respective pH may be determined in a routine way by a person skilled in the art.

The expression “the process of the invention for preparing 1,2-pentanediol according to the first aspect of the present invention is carried out at a pH of from 5.3 to 10.3, more preferably from 6.2 to 10.2, most preferably at a pH of 7.6” means, in the case of the process of the invention for preparing 1,2-pentanediol according to the first aspect of the present invention being carried out under autoclave conditions, that a base B1 is added before the reaction in such an amount that the pH of from 5.3 to 10.3, more preferably from 6.2 to 10.2, most preferably 7.6, can be measured in the reaction solution immediately after the end of the reaction. For the purposes of the invention, “immediately after the end of the reaction” means the point in time at which the proportion of 1,2-pentanediol no longer changes.

As base B1, it is in principle possible to use any alkaline substance by which the desired pH may be set; in particular, the base may be selected from the group consisting of alkali metal acetates, alkaline earth metal acetates, alkali metal carbonates, alkaline earth metal carbonates, alkali metal hydrogencarbonates, alkaline earth metal hydrogencarbonates, alkali metal hydroxides, alkaline earth metal hydroxides, and the base may preferably be selected from the group consisting of alkali metal acetates, alkaline earth metal acetates, alkali metal carbonates, alkaline earth metal carbonates, and the base may even more preferably be selected from the group consisting of alkali metal acetates, alkali metal carbonates; the base may particularly preferably be selected from the group consisting of sodium carbonate and sodium acetate, and the base may most preferably be sodium carbonate.

The process of the invention for preparing 1,2-pentanediol according to the first aspect of the present invention may also be conducted with addition of further organic compounds, which may be selected from the group consisting of sodium dicyanamide, 1-butyl-3-methylimidazolium dicyanamide, N-butyl-3-methylpyridinium dicyanamide, 1-butyl-1-methylpyrrolidinium dicyanamide, preferably 1-butyl-3-methylimidazolium dicyanamide. Even with addition of such organic compounds, the process of the invention according to the first aspect of the invention still displays surprisingly good yields. However, it has been found that the process of the invention according to the first aspect of the invention may result in best yields when no such addition is made. The process of the invention according to the first aspect of the present invention may therefore preferably be carried out without addition of organic compounds selected from the group consisting of sodium dicyanamide, 1-butyl-3-methylimidazolium dicyanamide, N-butyl-3-methylpyridinium dicyanamide, 1-butyl-1-methylpyrrolidinium dicyanamide.

The mixing of the reactants (furfuryl alcohol, H₂), which can, for example, be achieved by a particular stirring speed, can be effected in any way known to those skilled in the art.

The process of the invention is particularly suitable for the conversion of furfuryl alcohol into 1,2-pentanediol.

The Ru/AlOₓ catalyst system may be produced by supporting ruthenium on the aluminum oxide support. The ruthenium may be supported on the aluminum oxide support by impregnation, coating, deposition by coprecipitation or other suitable processes such as spray deposition. The Ru/AlOₓ catalyst system may preferably be produced by impregnating the aluminum oxide support with ruthenium. This may be effected by bringing the aluminum oxide support into contact with a ruthenium salt solution and depositing the ruthenium on the aluminum oxide support by spray treatment or by means of pH-controlled coprecipitation.

In one preferred mode of operation, the Ru/AlOₓ catalyst system may be obtained by a process for producing an Ru/AlOₓ catalyst system. This process comprises:

a) producing a mixture (i) comprising a ruthenium salt solution (ii) and an aqueous suspension of aluminum oxide (iii);
b) bringing the mixture (i) to a temperature in the range from greater than 0° C. to less than 100° C. and a pH of 0-14;
c) separating off the Ru/AlOₓ catalyst system by filtering the mixture (i);
d) drying the Ru/AlOₓ catalyst system separated off in c).

The present invention also provides an Ru/AlOₓ catalyst system obtained by the process for producing an Ru/AlOₓ catalyst system.

In a) of the process for producing an Ru/AlOₓ catalyst system, a mixture (i) comprising a ruthenium salt solution (ii) and an aqueous suspension of aluminum oxide (iii) is firstly produced. This can be carried out in any way known to those skilled in the art, but is typically carried out by mixing a ruthenium salt solution (ii) and an aqueous suspension of aluminum oxide (iii).

For the purposes of the invention, the ruthenium salt solution (i) may be any ruthenium-containing, preferably aqueous solution of a ruthenium salt. The ruthenium salt used for the purposes of the invention may be, in particular, selected from the group consisting of ruthenium carbonate [Ru(CO₃)₃]; ruthenium carboxylates such as ruthenium(II, III) μ-oxoacetate [(CH₃CO₂)₇Ru₃O-3H₂O]; ruthenium carbonyls; ruthenium halides such as ruthenium bromide (RuBr₃), ruthenium chloride (RuCl₃), ruthenium chloride hydrate (RuCl₃-xH₂O), ruthenium iodide (RuI₃); ruthenium nitrates such as Ru(NO₃)₃-xH₂O; ruthenium oxides such as RuO₂ and ruthenium(IV) oxide hydrate (RuO₂-xH₂O); ruthenium nitrosyl nitrates such as ruthenium nitrosyl nitrate [Ru(NO)(NO₃)ₓ(OH)_{y}, where x=1, 2, 3; y=0, 1, 2; and x+y=3]; ruthenium chloro complexes; ruthenium amine complexes, ruthenium nitrite complexes. In a preferred embodiment, the ruthenium salt may be ruthenium nitrosyl nitrate [Ru(NO)(NO₃)ₓ(OH)_{y}, where x=1, 2, 3; y=0, 1, 2; and x+y=3], with particular preference being given to ruthenium nitrosyl nitrate Ru(NO)(NO₃)₃.

As aqueous suspension of aluminum oxide (iii), it may be possible to use any suspension which comprises aluminum oxide, preferably Al₂O₃, more preferably γ-Al₂O₃.

In b) of the process for producing an Ru/AlOₓ catalyst system, the mixture (i) may be brought to a temperature in the range from greater than 0° C. to less than 100° C. and a pH of 0-14. This encompasses any procedure by which a mixture (i) comprising a ruthenium salt solution and an aqueous suspension of aluminum oxide having a temperature in the range from greater than 0° C. to less than 100° C. and a pH of 0-14 may be obtained. For this purpose, the ruthenium salt solution (ii) and/or the aqueous suspension of aluminum oxide (iii) may, for example, be brought to the appropriate temperature and/or the appropriate pH before the ruthenium salt solution (ii) and the aqueous suspension of aluminum oxide (iii) are mixed, so that the resulting mixture (i) has, without further action, the temperature in the desired range and the pH in the desired range. As an alternative, the ruthenium salt solution (ii) and an aqueous suspension of aluminum oxide (iii) may firstly be mixed and a temperature in the range from greater than 0° C. to less than 100° C. and a pH of 0-14 may then be set in the mixture (i).

For the purposes of the invention “temperature in the range from greater than 0° C. to less than 100° C.” preferably means a temperature of from 50° C. to less than 100° C., preferably from 55° C. to 75° C., more preferably 60° C.

In the embodiment according to the invention in which the ruthenium salt solution (ii) and an aqueous suspension of aluminum oxide (iii) are firstly mixed and a temperature in the range from greater than 0° C. to less than 100° C. and a pH of 0-14 are then set in the mixture (i), the mixture is preferably brought to a temperature in the range from greater than 0° C. to less than 100° C. by heating for a time of from 30 min to 300 min, preferably from 90 min to 240 min, more preferably from 120 min to 200 min, particularly preferably over a period of 180 min. In this embodiment, preference is also given to setting an alkaline pH of from greater than 7.0 to 14.0, preferably from greater than 7.0 to 10.0, more preferably from 7.5 to 8.5 and particularly preferably 8.0.

An acidic pH may be set using any organic or inorganic acid, in particular hydrohalic acids, preferably HCl, sulphuric acid, nitric acid, sulphurous acid, nitrous acid.

An alkaline pH may be achieved by addition of an appropriate amount of base B2. The base B2 can be added as solid or as solution to the mixture (i), the ruthenium salt solution (ii) or the aqueous suspension of aluminum oxide (iii), preferably to the mixture (i). A person skilled in the art will know what amount of the base B2 has to be added in order to set the desired pH.

For the purposes of the present invention, any organic or inorganic base may be used as base B2. In particular, the base B2 may be selected from the group consisting of alkaline earth metal hydroxides, alkali metal hydroxides, alkaline earth metal carbonates, alkali metal carbonates, alkaline earth metal hydrogencarbonates, alkali metal hydrogencarbonates, alkaline earth metal acetates, alkali metal acetates. The base B2 may preferably be selected from the group consisting of lithium hydroxide, sodium hydroxide, potassium hydroxide, lithium carbonate, sodium carbonate, potassium carbonate, lithium hydrogencarbonate, sodium hydrogencarbonate, potassium hydrogencarbonate, lithium acetate, sodium acetate, potassium acetate. The base B2 is particularly preferably selected from the group consisting of sodium carbonate, lithium hydroxide, lithium carbonate. The base B2 may especially preferably be sodium carbonate.

In a preferred embodiment, the process for producing an Ru/AlOₓ catalyst system is characterized in that a reducing agent (iv) is used. This can be realized by adding the reducing agent (iv) to the mixture (i) before b) or between b) and c), preferably between b) and c).

As reducing agent (iv), it may be possible to use any material which is able to reduce ruthenium. The reducing agent, in particular, is selected from the group consisting of hydrazine, borohydrides of the alkali metals, borohydrides of the alkaline earth metals, formates of the alkali metals, formates of the alkaline earth metals, hypophosphites of the alkali metals, hypophosphites of the alkaline earth metals, hydrogen, formic acid, formaldehyde. The reducing agent may preferably be selected from the group consisting of hydrazine, sodium formate, sodium borohydride, sodium hypophosphite, hydrogen, formic acid, formaldehyde. The reducing agent used may particularly preferably be formaldehyde, very particularly preferably as aqueous solution.

The reducing agent may preferably be added to the mixture (i) between b) and c). In this preferred embodiment of the process for producing an Ru/AlOₓ catalyst system, particular preference is given to the mixture being stirred for another 30-90 min, very particularly preferably 60 min, at temperature after addition of the reducing agent.

The pressure in a) and b) is not subject to any restrictions; in particular, the pressure may be atmospheric pressure (1 bar).

In c) of the process for producing an Ru/AlOₓ catalyst system, the mixture (i) obtained after b) of the process for producing an Ru/AlOₓ catalyst system and optional addition of the reducing agent after b) is filtered so as to obtain the Ru/AlOₓ catalyst system. Filtration may be achieved by filtration methods known to those skilled in the art, in particular methods such as decantation, filtration, centrifugation, vacuum filtration, and pressure filtration. In this way, the Ru/AlOₓ catalyst system may be isolated from the mixture (i).

In d) of the process for producing an Ru/AlOₓ catalyst system, the Ru/AlOₓ catalyst system obtained in c) is dried. This usually takes place under reduced pressure and at a temperature below 120° C., preferably below 100° C., particularly preferably at 80° C. For the purposes of the invention, “reduced pressure” means pressures below 1 bar, preferably below 0.8 bar, more preferably below 0.1 bar, particularly preferably below 0.02 bar, most preferably below 0.005 bar.

The content of ruthenium in the Ru/AlOₓ catalyst system after d) of the process for producing an Ru/AlOₓ catalyst system may be controlled by reacting the appropriate amount of ruthenium salt with the appropriate amount of aluminum oxide in a). This is within the capabilities of a person skilled in the art. For example, an Ru/AlOₓ catalyst system having an appropriate ruthenium content of 5% by weight may be produced by adding 5 g of ruthenium in the form of 15.68 g of ruthenium nitrosyl nitrate Ru(NO)(NO₃)₃ to an aqueous suspension of 95 g of aluminum oxide in a). The reducing agent may be added in a two-fold molar excess, preferably in an equimolar amount, based on the molar amount of ruthenium in the component (ii).

In a very particularly preferred embodiment of the present invention, the process for producing an Ru/AlOₓ catalyst system comprises:

a) producing a mixture (i) comprising a ruthenium salt solution (ii) and an aqueous suspension of aluminum oxide (iii);
b1) setting a pH of 7.5-8.5 with addition of a base B2;
b2) bringing the mixture (i) to a temperature in the range 55-75° C. by heating for a time of from 120 min to 200 min, particularly preferably 180 min;
b3) adding a reducing agent, preferably formaldehyde, to the mixture (i);
c) separating off the Ru/AlOₓ catalyst system by filtering the mixture (i);
d) drying the Ru/AlOₓ catalyst system separated off in step c).

In a second aspect, the present invention provides a process for preparing 1,2-pentanediol, comprising reacting furfuryl alcohol with hydrogen in the presence of an Ru/AC catalyst system.

For the purposes of the invention, the expression “Ru/AC catalyst system” is used as an abbreviation for “a catalyst system comprising (I) ruthenium and (II) a support composed of activated carbon on which the ruthenium is supported”.

The content of ruthenium in the Ru/AC catalyst system may be, in particular, in the range 0.01-30% by weight, preferably in the range 0.01-20% by weight, particularly preferably in the range 0.5-10.5% by weight, very particularly preferably in the range 5-10% by weight, most preferably 5% by weight, based on the total weight of the Ru/AC catalyst system. The values are measured after drying of the Ru/AC catalyst system at 80° C. under reduced pressure to constant weight. The content of ruthenium based on the total weight of the Ru/AC catalyst system can be determined in accordance with DIN51009.

It has surprisingly been found that use of the Ru/AC catalyst system in the process of the invention for preparing 1,2-pentanediol makes it possible to prepare the product 1,2-pentanediol in a surprisingly high selectivity compared to conventional methods, such as described, for example, by Zhang et al. [49].

The total BET surface area of the Ru/AC catalyst system may be in the range from 300 to 2000 m²/g of Ru/AC catalyst system, in particular from 500 to 1500 m²/g of Ru/AC catalyst system, preferably from 800 to 1100 m²/g of Ru/AC catalyst system. The total BET surface area is determined in accordance with DIN ISO 9277.

The pore volume of the Ru/AC catalyst system may be from 0.3 to 2.0 ml/g of Ru/AC catalyst system, preferably from 0.5 to 1.8 ml/g of Ru/AC catalyst system, particularly preferably from 0.6 to 1.5 ml/g of Ru/AC catalyst system. The pore volume is determined in accordance with DN ISO 9277.

The particle size distribution of the Ru/AC catalyst system may typically be d10=2-10 μm; d50=15-30 μm; d90=50-100 μm. According to the present invention, “d10=2-10 μm” means that 10% of the Ru/AC catalyst system has a particle size of 2-10 μm or less; “d50=15-30 μm” means that 50% of the Ru/AC catalyst system has a particle size of 15-30 μm or less; and “d90=50-100 μm” means that 90% of the Ru/AC catalyst system has a particle size of 50-100 μm or less.

In the process of the invention for preparing 1,2-pentanediol according to the second aspect of the invention, the temperature is in principle not subject to any restrictions; thus, for example, a temperature of greater than or equal to 100° C. may be set. The process of the invention for preparing 1,2-pentanediol according to the second aspect of the invention may be preferably carried out at a temperature of from 100° C. to 280° C., particularly preferably at a temperature of from 150 to 260° C. It has been found to be very particularly advantageous and it is therefore very particularly preferred for the process of the invention for preparing 1,2-pentanediol according to the second aspect of the invention to be carried out at a temperature of from 200° C. to 240° C.

The process of the invention for preparing 1,2-pentanediol according to the second aspect of the invention may preferably be conducted out under autoclave conditions. For the purposes of the invention, “autoclave conditions” describe pressures of equal to or greater than 10 bar, preferably pressures in the range from 10 to 200 bar, particularly preferably pressures in the range from 50 to 150 bar, most preferably a pressure of 100 bar.

The process of the invention for preparing 1,2-pentanediol according to the second aspect of the present invention may be conducted in any desired solvent. In particular, solvents selected from the group consisting of water, ethanol, tetrahydrofuran, 1,4-dioxane may be used in this context. Particular preference may be given to using water as solvent.

The process of the invention according to the second aspect of the present invention may be conducted batchwise or continuously.

In the process of the invention for preparing 1,2-pentanediol according to the second aspect of the present invention, any gas which comprises free hydrogen and is free of additives which could suppress the process, for example carbon monoxide, may be used as hydrogen.

The amount of Ru/AC catalyst system used in the process of the invention for preparing 1,2-pentanediol according to the second aspect of the present invention is not subject to any particular restrictions and may be in the range 0.01-30% by weight, preferably 0.5-30% by weight, particularly preferably 1-20% by weight, very particularly preferably 1-10% by weight, in each case based on the weight of the furfuryl alcohol used.

In addition, it has surprisingly been found that particularly good selectivities to 1,2-pentanediol are obtained when the process of the invention for preparing 1,2-pentanediol according to the second aspect of the present invention is carried out at a pH of from 5.3 to 10.3, more preferably from 6.2 to 10.2, most preferably at a pH of 7.6. This may be achieved by adding a base B1 before the reaction. The amount of the base necessary for achieving the respective pH may be determined by a person skilled in the art in a routine way.

The expression “the process of the invention for preparing 1,2-pentanediol according to the second aspect of the present invention is carried out at a pH of from 5.3 to 10.3, more preferably from 6.2 to 10.2, most preferably at a pH of 7.6” means, when the process of the invention for preparing 1,2-pentanediol according to the second aspect of the present invention is carried out under autoclave conditions, that a base B1 is added before the reaction in such an amount that the pH of from 5.3 to 10.3, more preferably from 6.2 to 10.2, most preferably 7.6, can be measured in the reaction solution immediately after the end of the reaction. For the purposes of the invention, “immediately after the end of the reaction” means the point in time at which the proportion of 1,2-pentanediol no longer changes.

The base B1 is defined above.

The Ru/AC catalyst system is produced by supporting the ruthenium on the activated carbon support. The ruthenium may be supported on the activated carbon support by impregnation, coating, deposition by coprecipitation or other suitable processes such as spray deposition. The Ru/AC catalyst system may preferably be produced by impregnating the activated carbon support with ruthenium. This may be achieved by bringing the activated carbon support into contact with a ruthenium salt solution and depositing the ruthenium on the activated carbon support by spray treatment or by means of pH-controlled coprecipitation.

In a preferred embodiment, the Ru/AC catalyst system is obtained by a process, comprising:

a) producing a mixture (i) comprising a ruthenium salt solution (ii) and an aqueous suspension of activated carbon (iii);
b) bringing the mixture (i) to a temperature in the range from greater than 0° C. to less than 100° C. and a pH of 0-14;
c) separating off the Ru/AC catalyst system by filtering the mixture (i).

The present invention also provides an Ru/AC catalyst system obtained by the process for producing an Ru/AC catalyst system.

In a) of the process for producing an Ru/AC catalyst system, a mixture (i) comprising a ruthenium salt solution (ii) and an aqueous suspension of activated carbon (iii) is firstly produced. This may be conducted in any way known to those skilled in the art, but may be conducted by mixing a ruthenium salt solution (ii) and an aqueous suspension of activated carbon (iii).

For the purposes of the invention, the ruthenium salt solution (i) may be any ruthenium-containing, preferably aqueous solution of a ruthenium salt. The ruthenium salt used for the purposes of the invention may be selected from the group consisting of ruthenium carbonate [Ru(CO₃)₃]; ruthenium carboxylates such as ruthenium(II, III) μ-oxoacetate [(CH₃CO₂)₇Ru₃O-3H₂O]; ruthenium carbonyls; ruthenium halides such as ruthenium bromide (RuBr₃), ruthenium chloride (RuCl₃), ruthenium chloride hydrate (RuCl₃-xH₂O), ruthenium iodide (RuI₃); ruthenium nitrates such as Ru(NO₃)₃-xH₂O; ruthenium oxides such as RuO₂ and ruthenium(IV) oxide hydrate (RuO₂-xH₂O); ruthenium nitrosyl nitrates such as ruthenium nitrosyl nitrate [Ru(NO)(NO₃)ₓ(OH)_{y}, where x=1, 2, 3; y=0, 1, 2; and x+y=3]; ruthenium chloro complexes; ruthenium amine complexes, ruthenium nitrite complexes. In a preferred embodiment, the ruthenium salt is ruthenium nitrosyl nitrate [Ru(NO)(NO₃)ₓ(OH)_{y}, where x=1, 2, 3; y=0, 1, 2; and x+y=3], with particular preference being given to ruthenium nitrosyl nitrate Ru(NO)(NO₃)₃.

As aqueous suspension of activated carbon (ii), it is possible to use any suspension which comprises activated carbon. For the purposes of the invention “activated carbon” is a term with which a person skilled in the art will be familiar and refers to an amorphous material which preferably contains more than 90% of carbon and has a highly porous structure, an internal surface area of preferably 300-2000 m²/g of carbon and a density in the range of preferably 0.2-0.6 g/cm³. Possible activated carbons for the purposes of the invention include chemically activated carbon, physically activated carbon, soot, carbon black, carbon nanotubes, aerogels, preferably carbon black.

In b) of the process for producing an Ru/AC catalyst system, the mixture (i) is brought to a temperature in the range from greater than 0° C. to less than 100° C. and a pH of 0-14. This encompasses any procedure by which a mixture (i) comprising a ruthenium salt solution and an aqueous suspension of activated carbon having a temperature in the range from greater than 0° C. to less than 100° C. and a pH of 0-14 is obtained. For this purpose, the ruthenium salt solution (ii) and/or the aqueous suspension of activated carbon (iii) may be brought to the appropriate temperature and/or the appropriate pH before the ruthenium salt solution (ii) and the aqueous suspension of activated carbon (iii) may be mixed so as to obtain the mixture (i) having the desired temperature and the desired pH. As an alternative, the ruthenium salt solution (ii) and an aqueous suspension of activated carbon (iii) can firstly be mixed and a temperature in the range from greater than 0° C. to less than 100° C. and a pH of 0-14 can then be set in the mixture (i).

For the purposes of the invention, “temperature in the range from greater than 0° C. to less than 100° C.” preferably means a temperature of from 50° C. to less than 100° C., preferably from 55° C. to 75° C., more preferably 60° C.

In the embodiment of the invention in which the ruthenium salt solution (ii) and an aqueous suspension of activated carbon (iii) are firstly mixed and a temperature in the range from greater than 0° C. to less than 100° C. and a pH of 0-14 are then set in the mixture (i), the mixture may be brought to a temperature in the range from greater than 0° C. to less than 100° C. by heating for a time of from 30 min to 300 min, preferably from 90 min to 240 min, more preferably from 120 min to 200 min, particularly preferably over a period of 180 min. In this embodiment, an alkaline pH of from greater than 7.0 to 14.0, preferably from greater than 7.0 to 10.0, more preferably from 7.5 to 8.5 and particularly preferably of 8.0, may be set.

An acidic pH may be set using any organic or inorganic acid, in particular hydrohalic acids, preferably HCl, sulphuric acid, nitric acid, sulphurous acid, nitrous acid.

An alkaline pH may be achieved by adding an appropriate amount of base B2 to the respective components (i), (ii), (iii). The base can be added as solid or as a solution. A person skilled in the art will know what amount of base B2 has to be added in order to set the desired pH.

In a preferred embodiment of the process for producing an Ru/AC catalyst system, an alkaline pH of the mixture (i), more preferably a pH in the range from greater than 7.0 to 14.0, preferably from greater than 7.0 to 10.0, more preferably from 7.5 to 8.5 and particularly preferably 8.0, is set in a).

For the purposes of the present invention, any organic or inorganic base may be used as base B2. In particular, the base B2 may be selected from the group consisting of alkaline earth metal hydroxides, alkali metal hydroxides, alkaline earth metal carbonates, alkali metal carbonates, alkaline earth metal hydrogencarbonates, alkali metal hydrogencarbonates, alkaline earth metal acetates, alkali metal acetates. The base B2 is preferably selected from the group consisting of lithium hydroxide, sodium hydroxide, potassium hydroxide, lithium carbonate, sodium carbonate, potassium carbonate, lithium hydrogencarbonate, sodium hydrogencarbonate, potassium hydrogencarbonate, lithium acetate, sodium acetate, potassium acetate. The base B2 is particularly preferably selected from the group consisting of sodium carbonate, lithium hydroxide, lithium carbonate. The base B2 may especially preferably be sodium carbonate.

In a preferred embodiment, the process for producing an Ru/AC catalyst system comprises a reducing agent (iv). The reducing agent (iv) may be added to the mixture (i) before b) or between b) and c), preferably between b) and c).

As reducing agent (iv), it is possible to use any material which is able to reduce ruthenium. The reducing agent may be selected from the group consisting of hydrazine, borohydrides of the alkali metals, borohydrides of the alkaline earth metals, formates of the alkali metals, formates of the alkaline earth metals, hypophosphites of the alkali metals, hypophosphites of the alkaline earth metals, hydrogen, formic acid, formaldehyde. The reducing agent may preferably be selected from the group consisting of hydrazine, sodium formate, sodium borohydride, sodium hypophosphite, hydrogen, formic acid, and formaldehyde. The reducing agent used may particularly preferably be formaldehyde, very particularly preferably as aqueous solution.

The reducing agent is preferably added to the mixture (i) between b) and c). In this preferred embodiment of the process for producing an Ru/AC catalyst system, particular preference may be given to the mixture being stirred for another 30-90 min, very particularly preferably 60 min, at temperature after addition of the reducing agent.

The pressure a) and b) is not subject to any restrictions and may be at atmospheric pressure (1 bar).

In c) of the process for producing an Ru/AC catalyst system, the mixture (i) obtained after b) of the process for producing an Ru/AC catalyst system and optional addition of the reducing agent after b) is filtered so as to obtain the Ru/AC catalyst system. Filtration may be achieved by filtration methods known to those skilled in the art, in particular methods such as decantation, filtration, centrifugation, vacuum filtration, pressure filtration. In this way, the Ru/AC catalyst system can be isolated from the mixture (i).

The content of ruthenium in the Ru/AC catalyst system after d) of the process for producing an Ru/AC catalyst system may be controlled by reacting the appropriate amount of ruthenium salt with the appropriate amount of activated carbon in a). This is within the capabilities of a person skilled in the art. For example, an Ru/AC catalyst system having an appropriate ruthenium content of 5% by weight can be produced by adding 5 g of ruthenium in the form of 15.68 g of ruthenium nitrosyl nitrate Ru(NO)(NO₃)₃ to an aqueous suspension of 95 g of activated carbon in step a). The reducing agent may be added in a two-fold molar excess, preferably in an equimolar amount, based on the molar amount of ruthenium in the component (ii).

In a very particularly preferred embodiment of the present invention the process for producing an Ru/AC catalyst system, comprises:

a) producing a mixture (i) comprising a ruthenium salt solution (ii) and an aqueous suspension of activated carbon (iii);
b1) setting a pH of 7.5-8.5 with addition of a base B2;
b2) bringing the mixture (i) to a temperature in the range 55-75° C. by heating for a time of from 120 min to 200 min, particularly preferably 180 min;
b3) adding a reducing agent, preferably formaldehyde, to the mixture (i);
c) separating off the Ru/AC catalyst system by filtering the mixture (i).

In a third aspect, the present invention provides a process for preparing 1,2-pentanediol, comprising reacting furfuryl alcohol with hydrogen in the presence of a Pt(IV) oxide catalyst.

For the purposes of the present invention, “Pt(IV) oxide catalyst” means an Adams-type catalyst and refers to Pt(IV) oxide.

It has surprisingly been found that use of the Pt(IV) oxide catalyst in the process of the invention for preparing 1,2-pentanediol makes it possible to prepare the product 1,2-pentanediol with a surprisingly high selectivity.

In the process of the invention for preparing 1,2-pentanediol according to the third aspect of the invention, the temperature is in principle not subject to any restrictions. However, the process may be conducted at a temperature in the range from greater than 0° C. to less than 100° C., more preferably in the range from 5° C. to 60° C., even more preferably in the range from 20° C. to 55° C.

The pressure in the process of the invention for preparing 1,2-pentanediol according to the third aspect of the invention is in principle not subject to any restrictions and may be 1-10 bar, preferably 1-5 bar, particularly preferably 2 bar.

The process of the invention according to the third aspect of the present invention can be carried out in any desired solvent. In particular, solvents selected from the group consisting of acetic acid, water and ethanol may be employed. Particularly good selectivities to 1,2-pentanediol have been observed when ethanol was used as solvent. Accordingly, ethanol may be preferably chosen as solvent in the process of the invention according to the third aspect of the present invention.

Having generally described the present invention, a further understanding can be obtained by reference to certain specific examples which are provided herein for purposes of illustration only, and are not intended to be limiting unless otherwise specified.

### EXAMPLES

### 1. Chemicals Used

The following chemicals were used:

Furfuryl alcohol (Sigma Aldrich, ≧98%)

### Solvents:

1,4-Dioxane (Merck, ≧99%), acetic acid (Sigma Aldrich, ≧99.5%), ethanol (Roth, ≧99.8%), THF (Sigma Aldrich, ≧99.9%), water (VWR, HiPerSolv CHROMANORM).
Acids and bases:
Sodium acetate (F. Klasovsky, ACS grade), sodium carbonate (Sigma Aldrich, ≧99.5%), 2N hydrochloric acid (Roth).
Metal precursor compounds:
Copper(II) nitrate trihydrate (Merck, ≧99.5%), nickel(II) nitrate hexahydrate (Aldrich, 99.999%), tetraammineplatinum(II) nitrate (Alfa Aesar, 99.99%), zinc nitrate hexahydrate
(Sigma Aldrich, 98%), tin(II) chloride (Alfa Aesar, ≧99%).
For calibration of GC and GC-MS:
1,2-Butanediol (Fluka, ≧98%), cyclopentanol (Aldrich, 99%), cyclopentanone (Sigma Aldrich, ≧99%), 1,2-hexanediol (Aldrich, 98%), 2-methylfuran (Aldrich, 99%), 1-pentanol
(Sigma Aldrich, ≧99%), 1,2-pentanediol (Aldrich, 96%), 1,5-pentanediol (Fluka, ≧97%), tetrahydrofurfuryl alcohol (Fluka, ≧98%).
Ionic liquids:
1-Butyl-3-methylimidazolium dicyanamide (Merck), N-butyl-3-methylpyridinium dicyanamide (Merck), 1-butyl-1-methylpyrrolidinium dicyanamide (Merck).

### Gases:

Argon (Linde, ≧99.999%), hydrogen (Linde, ≧99.999%).

### Miscellaneous:

Sodium dicyanamide (Aldrich, 96%)

### Catalysts:

TABLE 6
Catalysts tested for the hydrogenolysis of furfuryl alcohol.

| Catalyst designation | Manufacturer | Composition |
|---|---|---|
| Al1 | Produced in-house | 5% Ru/Al₂O₃ |
| | (Section 2.11) | |
| Al2 | Produced in-house | 5% Ru/AC |
| | (Section 2.12) | |
| Al3 | Produced in-house | 5% Ru/AC |
| | (Section 2.12) | |
| RR506 | Sudzucker | 1% Ru/Al₂O₃ |
| Y40753-BS | KataLeuna | 1% Ru/Al₂O₃ |
| FS384 | Produced in-house | 1% Ru on porous glass |
| Pt-JA-024 | Produced in-house | Pt/Polyaniline |
| Pt-JA-026 | Produced in-house | 10% Pt/C |
| Pt-JA-023 | Produced in-house | Pt(IV) Oxide |
| Pt-JA-021 | Produced in-house | Pt(IV) Oxide |
| Heraeus | Heraeus | Pt(IV) Oxide |
| Sigma Aldrich | Sigma Aldrich | Pt(IV) Oxide |

### 2. General Experimental Methods

The catalysts were used in the form of fine powders. If they were present as shaped bodies, they were firstly broken up in a mortar and sieved, with only the finest fraction having particle sizes of ≦200 μm being used for the hydrogenolysis.

### General Experimental Set-Up

The stirring autoclave (Parr Instruments) was made of stainless steel and had a capacity of 300 ml. A 500 ml gas tank was connected to the reactor via a pressure reducer and was supplied via a gas feed line system both with argon (≦80 bar) and with hydrogen (≦200 bar). An off gas valve served to depressurize the reactor after the end of the experiment. A gas introduction stirrer with a maximum stirring speed of 1600 rpm was used for mixing. A thermostat-controlled heating jacket by means of which temperatures up to 280° C. could be set served for heating the reaction mixture. Samples could be taken via an off take tap during the reaction. Furthermore, a supply tank to which hydrogen and argon could likewise be introduced from the gas tank was connected to the reactor for the introduction of the starting material. The pressure was measured by a manometer and the temperature in the gas and liquid phases was measured separately by means of thermocouples. The courses of pressure and temperature are stored digitally and could be accessed by computer.

2.1 General Experimental Method for Reacting Furfuryl Alcohol with Hydrogen in the Presence of an Ru/AlOₓ Catalyst

An aqueous suspension of solvent and catalyst and also any additives such as base or ionic liquid were placed in the autoclave. The reaction apparatus was closed and a stirring speed of 1000 rpm was set. The reactor was flushed three times with 10 bar of argon and once with 10 bar of hydrogen. The hydrogen pressure was subsequently set so that the expected pressure on reaching the reaction temperature was 10-20 bar below the reaction pressure. The contents of the autoclave were heated to reaction temperature over a period of 30-60 minutes. As soon as this has been reached, furfuryl alcohol or a solution of furfuryl alcohol was introduced quickly via the supply tank. Further hydrogen was continually supplied via the gas tank so as to keep the reaction pressure constant. If samples were to be taken during the reaction, the off take line should be flushed beforehand with 1-2 ml of reaction mixture and 0.5-1 ml of sample then taken. When the hydrogen pressure in the gas tank no longer decreased, the heating jacket was removed and the reactor was cooled in air to room temperature. After the reaction mixture had been taken off, the reactor was thoroughly cleaned and baked at 150° C. under 30 bar of argon for 60 minutes.

The compositions of the liquid phases initially placed in the autoclave and in the supply tank are listed in Table 7 for various starting material concentrations c(FFOH) and different solvents. The reaction volume at room temperature was always 100 ml.

TABLE 7
Composition of the liquid phases initially
placed in the autoclave and supply tank.

| c(FFOH) [g/100 ml | | Composition in | Composition in |
|---|---|---|---|
| of reaction solution] | Solvent | autoclave | supply tank |
| 7.46 | Water | 86.8 ml of water | 7.46 g of FFOH, |
| | | | 6.6 ml of H₂O |
| 20 | Water | 75.0 ml of water | 20.0 g of FFOH, |
| | | | 7.0 ml of H₂O |
| 40 | Water | 64.6 ml of water | 40.0 g of FFOH |
| 40 | Ethanol | 64.0 ml of ethanol | 40.0 g of FFOH |
| 40 | THF | 63.8 ml of THF | 40.0 g of FFOH |
| 40 | 1,4- | 64.0 ml of 1,4- | 40.0 g of FFOH |
| | Dioxane | Dioxane | |

2.2 General Experimental Method for Reacting Furfuryl Alcohol with Hydrogen in the Presence of a Pt(IV) Oxide Catalyst (Pt-JA-023)

A solution of 20 g of FFOH in 81 ml of ethanol was placed together with 0.5 g of catalyst Pt-JA-023 in the autoclave. The reaction apparatus was closed and a stirring speed of 1000 rpm was set. The reactor was flushed three times with 10 bar of argon and once with 10 bar of hydrogen. A hydrogen pressure of 20 bar was set in the reactor and kept constant by continually supplying further hydrogen from the gas tank. After 10 minutes, the contents of the autoclave were heated to 50° C. and after a further 30 minutes to 75° C. Another 45 minutes later, the reaction pressure is finally increased to 60 bar and the reaction temperature was increased to 180° C. After a total reaction time of 100 minutes, the heating jacket was removed and the reaction mixture was cooled in air to room temperature. The contents of the reactor were taken off and the reaction apparatus was thoroughly cleaned.

2.3 General Experimental Method for Reacting Furfuryl Alcohol with Hydrogen in the Presence of a Pt(IV) Oxide Catalyst (Heraeus) in Ethanol

A suspension of 0.5 g of Pt(IV) oxide catalyst from Heraeus in 71 ml of ethanol was placed in the autoclave. The reaction apparatus was closed and a stirring speed of 1000 rpm was set. The reactor was flushed three times with 10 bar of argon and once with 10 bar of hydrogen. After a hydrogen pressure of 6 bar had been set in the reactor via the gas tank, the reactor was heated to 75° C. A hydrogen pressure of 10 bar was subsequently set via the gas tank and the contents of the autoclave were stirred for 30 minutes. The heating jacket was removed and the reactor was cooled in air until the suspension had reached room temperature. The reactor was depressurized to 1.5 bar via the off gas valve. A solution of 10 g of FFOH in 20 ml of ethanol was then introduced quickly via the supply tank. The hydrogen pressure was set to 2 bar and kept constant by continually supplying further hydrogen from the gas tank. 170 minutes after the commencement of the reaction, the reaction temperature was increased to 50° C. After a total reaction time of 300 minutes, the heating jacket was finally removed and the reactor was cooled in air to room temperature. The contents of the reactor were taken off and the reaction apparatus was thoroughly cleaned.

2.4 General Experimental Method for Reacting Furfuryl Alcohol with Hydrogen in the Presence of a Pt(IV) Oxide Catalyst (Heraeus) in Acetic Acid

A suspension of 0.5 g of catalyst in 71 ml of acetic acid was placed in the autoclave. The reaction apparatus was closed and a stirring speed of 1000 rpm was set. The reactor was flushed three times with 10 bar of argon and once with 10 bar of hydrogen. After a hydrogen pressure of 8 bar had been set in the reactor via the gas tank, the contents of the reactor were heated to 75° C. A hydrogen pressure of 10 bar was subsequently set by the gas tank and the contents of the autoclave were stirred for 30 minutes. The heating jacket was removed and the reactor was cooled in air until the suspension had reached room temperature. The reactor was depressurized to 1.5 bar via the off gas valve. A solution of 10 g of FFOH in 20 ml of acetic acid was then introduced quickly via the supply tank and, at a reaction temperature of 50° C., the pressure was kept constant at 2 bar by supplying further hydrogen from the gas tank. 30 minutes after commencement of the reaction, the temperature was increased to 75° C. After a total reaction time of 150 minutes, the heating jacket was removed and the reactor was cooled in air. The contents of the reactor were taken off and the reaction apparatus was thoroughly cleaned.

2.5 General Experimental Method for Reacting Furfuryl Alcohol with Hydrogen in the Presence of a Pt(IV) Oxide Catalyst (Pt-JA-021; Sigma Aldrich) in Ethanol without Addition of Hydrochloric Acid

A suspension of 0.5 g of catalyst Pt-JA-021 or 0.5 g of Pt(IV) oxide catalyst from Sigma Aldrich in 71 ml of ethanol was placed in the autoclave. The reaction apparatus was closed and a stirring speed of 1000 rpm was set. The reactor was flushed three times with 10 bar of argon and once with 10 bar of hydrogen. After a hydrogen pressure of 6 bar had been set in the reactor via the gas tank, the contents of the reactor were heated to 75° C. A hydrogen pressure of 10 bar was subsequently set via the gas tank and the contents of the reactor were stirred for 30 minutes. If the reaction was to be carried out at room temperature, the heating jacket was removed and the reactor was cooled in air until the suspension has reached room temperature. The reactor was depressurized to 1.5 bar via the offgas valve. A solution of 10 g of FFOH in 20 ml of solvent was then introduced quickly via the supply tank. The reaction pressure was set and kept constant by continually supplying further hydrogen from the gas tank. When the pressure in the gas tank no longer decreased, the heating jacket was removed and the reactor was cooled in air to room temperature. The contents of the reactor were taken off and the reaction apparatus was thoroughly cleaned.

2.6 General Experimental Method for Reacting Furfuryl Alcohol with Hydrogen in the Presence of a Pt(IV) Oxide Catalyst (Pt-JA-021) in Ethanol with Addition of Hydrochloric Acid

The experiment was carried out in a manner analogous to that without addition of hydrochloric acid (section 2.5). Instead of placing the suspension composed of solvent and catalyst directly in the reactor, it was introduced together with 1 ml of 2N hydrochloric acid into a Teflon insert installed in the reactor.

2.7 General Experimental Method for Reacting Furfuryl Alcohol with Hydrogen in the Presence of a Pt(IV) Oxide Catalyst (Sigma Aldrich) in Ethanol with Addition of Hydrochloric Acid

A suspension of 0.5 g of catalyst, 10 g of FFOH, 91 ml of ethanol and 1 ml of 2N hydrochloric acid was placed in a Teflon insert. The reaction apparatus was closed and a stirring speed of 1000 rpm was set. The reactor was flushed three times with 10 bar of argon and once with 10 bar of hydrogen. The reactor was depressurized via the offgas valve until the desired reaction pressure had been reached. The pressure was kept constant during the reaction by continually supplying further hydrogen from the gas tank. When the pressure in the gas tank no longer decreased, the heating jacket was removed and the reactor was cooled in air to room temperature. The contents of the reactor were taken off and the reaction apparatus was thoroughly cleaned.

2.8 General Experimental Method when Using Furan, THF and THFFOH as Starting Material

A suspension of 0.5 g of 5% Ru/Al₂O₃ (A11) and a solution of the starting material in 100 ml of water were placed in the autoclave. The reaction apparatus was closed and a stirring speed of 1000 rpm was set. The reactor was flushed three times with 10 bar of argon and once with 10 bar of hydrogen. The hydrogen pressure is subsequently set so that the expected pressure on reaching the reaction temperature is 10-20 bar below the reaction pressure. The contents of the reactor were heated to reaction temperature over a period of 30-60 minutes. After the reaction time had elapsed, the heating jacket was removed and the reactor was cooled in air to room temperature. After taking off the reaction mixture, the reactor was thoroughly cleaned and baked at 150° C. under 30 bar of argon for 60 minutes.

2.9 General Experimental Method for Reacting Furfuryl Alcohol with Hydrogen in the Presence of a Pt(IV) Oxide Catalyst—Experiment in a Glass Apparatus

A simple glass apparatus was used for the hydrogenolysis using Pt(IV) oxide catalyst (Sigma Aldrich) at atmospheric pressure.

### Experimental Set-Up

The reaction mixture was mixed in a 250 ml three-neck flask with a magnetic stirrer. It was ensured by means of a reflux condenser that very little solvent was liberated during the reaction. The temperature could be read off by means of a thermometer dipping into the reaction solution. Nitrogen or hydrogen, as desired, could be introduced into the reaction solution at a variable flow rate via a float-type flow meter.

### Experimental Procedure

A suspension consisting of 10 g of FFOH, 91 ml of ethanol, 1 ml of 2N hydrochloric acid and 0.5 g of Pt(IV) oxide (Sigma Aldrich) was placed in the three-neck flask. The flask was flushed with nitrogen for 10 minutes while stirring. Hydrogen was subsequently introduced at a volume flow rate of 270 cm³ per minute and the mixture was stirred at room temperature for 270 minutes.

2.10 General Experimental Method for Reacting Furfuryl Alcohol with Hydrogen in the Presence of a Pt(IV) Oxide Catalyst—Experiment in a Multibatch Plant

The experiments for comparing the catalyst performance with addition of various ionic liquids were carried out in a multibatch plant.

### Experimental Set-Up

The multibatch plant comprised five reactors made of stainless steel and each having a capacity of 40 ml. Mixing was ensured by means of a five-fold magnetic stirrer. Each reactor was preceded by a 75 ml gas tank which was made of stainless steel and could be supplied with argon or hydrogen at ≦100 bar via the gas feed line system. The individual reactors could be heated independently by means of a thermostat-controlled battery of heating blocks. Control and monitoring of the reaction temperature were computer-controlled.

### Experimental Procedure

A suspension composed of 4 g of FFOh, 6.1 ml of water, 100 mg of the 5% Ru/Al₂O₃ catalyst A11 and 5 μl of ionic liquid (or 2.5 mg of sodium dicyanamide) was placed in the reactor. The reactor was closed and connected to the plant. After flushing three times with 30 bar of argon and once with 50 bar of hydrogen, the reactor was pressurized to 85 bar with hydrogen and heated to 180° C. The pressure temporally reached up to 90 bar during heating and towards the end of the reaction dropped to about 75 bar. The experiment was stopped 100 minutes after the reaction temperature had been reached. For this purpose, the pressure line of the reactor was disconnected from the plant and the reactor was cooled to room temperature in a water bath. The reactor was depressurized to ambient pressure by carefully opening the needle valve and the reaction mixture is taken from the reactor.

### 2.11 Production of the 5% Ru/Al₂O₃ Catalyst A11

15.7 g of ruthenium nitrosyl nitrate in 500 ml of deionized water were added dropwise to a suspension of 95 g of gamma-aluminum oxide in 1500 ml of deionized water. The pH of the suspension was set to 8.0 by addition of Na₂CO₃. The suspension was heated to 60° C. over a period of 3 hours and subsequently stirred at 60° C. for a further one hour. An equimolar amount (equimolar based on ruthenium) of formaldehyde was then added as aqueous solution and the mixture was stirred for another one hour. The catalyst was separated off by filtration and dried to constant weight at 80° C. under reduced pressure. The ruthenium content of the catalyst corresponded to 5% by weight of ruthenium based on the total Ru/Al₂O₃ catalyst A11. The content of ruthenium can be checked by AAS/ICP (DIN51009).

The catalyst obtained was examined by X-ray powder diffraction. X-ray powder diffraction is a non-destructive analytical method for determining crystal forms (also phases) in powders or solids.

The principle of X-ray powder diffraction is as follows: when an X-ray beam impinges on an atom, it is scattered by the electrons thereof. If the atoms are, as in crystalline materials, arranged periodically, interference occurs at a particular angle (2θ) between the reflected direction and incident direction of the X-ray beam. This interference condition is dependent on the wavelength λ of the X-radiation used and the spacing d of the reflecting planes in the lattice and is described by the Bragg equation:

2d sin θ=nλ,

The intensity of the reflected X-ray beam is measured as a function of the Bragg scattering angle θ. The intensity is usually reported as a function of 2θ in the diffraction pattern.

Owing to the regular arrangement of the atoms on lattice planes in crystals, discrete reflections at characteristic lattice plane spacings are detected in the case of crystalline material.

The angle of the reflections is determined only by the geometry of the unit cell of the crystalline phase. The relative intensity ratio of the reflections observed, on the other hand, is modulated by the different scattering behaviour of the atoms as a function of the electron density (atomic number) and the position of the individual atoms in the unit cell.

The width at half height of the reflections indicates the crystal size of the phase examined.

### Sample Preparation

The sample was prepared as follows: a portion of the sample material supplied (typically in the range from 0.5 g to 2 g) was prepared in a 16 mm sample carrier by means of backloading. Here, the sample carrier was filled with the sample from the rear side in order to minimize the preferential orientation.

The sample which had been prepared in this way was analysed in a PANalytical X'Pert MPD Pro instrument under the following measurement parameters:

### Parameters:

XRD instrument: PANalytical Theta/Theta diffractometer
X-ray tube: LFF-Cu X-ray tube, Cu Kα, λ=0.1542 nm

### Excitation: 40 mA, 40 KV

### Detector: X'Celerator

Sample holder: Ø 16 mm
Rotation: Yes/1 revolution/s
2-Theta measurement range: 5°-100°

### Step (° 2Θ) 0.017°

Time per step: 40 s
n (see Bragg equation)=1
The following instruments were used:

### Instruments:

### PANalytical X'Pert MPD Pro

Test method: XRPD (X-ray powder diffraction)
Test procedure: SOP ROE-002
Evaluation was by means of the current version of PANalytical software HighScore Plus and current version of the ICDD databank with crystalline reference phases
The following peaks were found:

| Pos. | Relative |
|---|---|
| [°2Th.] | Intensity [%] |
| 18.3 | 8.69 |
| 18.8 | 13.92 |
| 20.33 | 8.8 |
| 31.37 | 51.95 |
| 32.68 | 28.16 |
| 32.825 | 82.43 |
| 35.09 | 12.34 |
| 36.758 | 56.2 |
| 38.97 | 30.89 |
| 39.8 | 32.12 |
| 40.63 | 10.43 |
| 43.359 | 16.21 |
| 44.85 | 64.42 |
| 45.47 | 47.23 |
| 46.54 | 28.33 |
| 47.61 | 32.26 |
| 50.77 | 11.05 |
| 51.5 | 9.98 |
| 57.48 | 8.68 |
| 59.97 | 17.46 |
| 62.38 | 11.05 |
| 62.848 | 10.72 |
| 64.05 | 13.91 |
| 65.7 | 12.04 |
| 66.52 | 56.88 |
| 67 | 34.05 |
| 67.4 | 100 |
| 73.5 | 6.88 |

The 5% Ru/Al₂O₃ catalyst A11 was mesoporous and had a pore diameter in the range from 2 nm to 50 nm, determined in accordance with DIN ISO 9277.

The total BET surface area of the 5% Ru/Al₂O₃ catalyst A11 was in the range from 100 to 150 m²/g of catalyst. The total BET surface area was determined in accordance with DIN ISO 9277.

The pore volume of the 5% Ru/Al₂O₃ catalyst A11 is 0.4 ml/g of catalyst. The pore volume was determined in accordance with DIN ISO 9277.

### 2.12 Production of the 5% Ru/AC Catalyst a12 and of the 5% Ru/C Catalyst A13

The 5% Ru/AC catalyst A12 and the 5% Ru/C catalyst A13 were produced in a manner analogous to that described for the catalyst A11 (section 2.11) with the difference that the appropriate amount of activated carbon was used instead of Al₂O₃ as support and no drying took place after filtration before the catalyst was utilized. The content of ruthenium could be checked by AAS/ICP after drying to constant weight at 80° C. under reduced pressure (DINS 1009).

### 2.13 Production of the [5:2] Ru—Zn, [5:2] Ru—Fe, [1:2] Ru—Cu and [1:2] Ru—Ni Catalysts on Al₂O₃

The preparation of the Ru—Zn, Ru—Fe, Ru—Cu and Ru—Ni catalysts on aluminum oxide was carried out by impregnation using the incipient wetness method. For this purpose, 0.9 ml of a solution of the metal precursor compound in water was added dropwise to 1 g of the 5% Ru/Al₂O₃ catalyst A11. The moist catalyst was dried overnight at 85° C. in a drying oven. Calcination was carried out in a heat treatment furnace under a stream of air of 130 cm³/min, with the catalyst being heated to 200° C. over a period of one hour and this temperature being kept constant for one hour. Cooling to room temperature was effected in air at the same volume flow. Reduction was carried out at a hydrogen flow of 270 cm³/min by heating to 250° C. over a period of one hour and subsequently keeping the temperature constant for one hour. The heat treatment reactor was cooled in air under a stream of hydrogen to 100° C. and subsequently under a stream of nitrogen to room temperature. The amounts of the individual metal precursor compounds used are shown in Table 8.

TABLE 8
Amounts of the metal precursor compounds used
for the preparation of bimetallic catalysts

| Second | | |
|---|---|---|
| metal | Precursor compound | Amount |
| Zn | Zinc(II) nitrate hexahydrate | 59.5 mg |
| Fe | Iron(III) nitrate nonahydrate | 77.2 mg |
| Cu | Copper(II) nitrate trihydrate | 239.2 mg |
| Ni | Nickel(II) nitrate hexahydrate | 287.9 mg |

### 2.14 Production of the [1:2] Ru—Pt Catalyst on Al₂O₂

The Ru—Pt/Al₂O₃ catalyst was produced by impregnation from supernatant solution of the metal precursor compound. For this purpose, a solution of 383 mg of tetraammineplatinum(II) nitrate in 2.7 ml of water was added dropwise to 1 g of the 5% Ru/Al₂O₃ catalyst A11. The suspension was stored in a closed vessel for 2 days. After decanting of the supernatant solution, the moist catalyst was dried overnight at 85° C. in a drying oven. Calcination and reduction were carried out in a manner analogous to the other bimetallic catalysts.

### 2.15 Production of the [17:2] Ru—Sn Catalyst on Al₂O₃

The Ru—Sn/Al₂O₃ catalyst was produced in situ by placing 10 mg of Sn(II) chloride together with the suspension of the catalyst in the autoclave. The experimental procedure was carried out as per the general method under section 2.1.

### 3. Analysis

A sample was taken from each hydrogenolysis experiment after the end of the reaction. This was filtered and the filtrate was diluted 1:10 with water. When high initial FFOH concentrations of ≧200 g/l were used, dilution was instead by a factor of 1:20. The diluted samples were analysed by GC and GC-MS in order to identify the reaction products and also determine conversion and selectivities.

To identify the reaction products, a GCMS-QP2010 SE with gas chromatograph GC-20120 Plus from SHIMADZU was used. A DB wax column having the specifications shown in Table 9 served as stationary phase.

TABLE 9
Specifications of the column used and analytical
method in GC and GC-MS studies

| Parameter | Value | |
|---|---|---|
| Length | 30 | m |
| Diameter | 0.25 | mm |
| Film thickness | 0.25 | μm |
| Start temperature | 50° | C. |
| Heating rate | 10° | C./min |
| Final temperature | 220° | C. |
| Hold time | 8 | min |
| Eluent | Helium | |
| Eluent flow | 1 | ml/min |

A gas chromatograph HP 6890 GC System from Hewlett Packard was used for determining conversions and selectivities. Column and method parameters are identical to the parameters shown in Table 9 for the GC-MS studies. A flame ionization detector was used for signal detection.

To identify the detected signals, aqueous solutions of furfuryl alcohol and the most important reaction products expected were examined and compared with GC-MS results for unambiguous assignment. Aqueous standard solutions of the respective compound in the concentration range from 0.5 g/l to 10 g/l were used for quantitative calibration. In the case of compounds for which no quantitative calibration was carried out, calibration factors of 0.05 mmol/l per peak area were assumed. The measured retention times and calibration factors are summarized in Table 10.

TABLE 10
Retention times and calibration factors

| | Retention | Calibration factor [mmol/l |
|---|---|---|
| Compound | time [min] | per peak area] |
| THF | 3.011 | not determined |
| 2-Methyltetrahydrofuran | 3.087 | not determined |
| 2-Methylfuran | 3.155 | not determined |
| 2-Pentanol | 5.198 | not determined |
| 1-Butanol | 5.438 | 0.0645 |
| Cyclopentanone | 5.990 | 0.0664 |
| 1-Pentanol | 6.667 | not determined |
| Cyclopentanol | 7.330 | 0.0598 |
| Cyclopentenone | 8.034 | not determined |
| Tetrahydrofurfuryl alcohol | 9.815 | 0.0468 |
| Furfuryl alcohol | 11.478 | 0.0565 |
| 1,2-Butanediol | 11.722 | 0.0649 |
| 2S,4R-Pentanediol | 11.838 | not determined |
| 1,2-Pentanediol | 12.819 | 0.0504 |
| 1,4-Pentanediol | 13.885 | not determined |
| 1,5-Hexanediol | 14.939 | not determined |
| 1,5-Pentanediol | 15.378 | 0.0455 |
| 4-Hydroxy-2- | 16.376 | not determined |
| cyclopentenone | | |

### 4. Results

### Evaluation of Catalyst Systems in Respect of Conversion and Selectivity of Furfuryl Alcohol to 1,2-pentanediol

Various catalyst systems were examined in respect of conversion of furfuryl alcohol and selectivity to 1,2-pentanediol. Firstly, supported ruthenium and platinum catalysts which require high pressures and temperatures in order to achieve good activity and selectivity were used. For the most selective of these catalysts, further experiments were carried out with variation of the reaction conditions and with addition of various second metals and ionic liquids. Secondly, platinum(IV) oxide catalysts which are active even under substantially milder conditions in organic solution were investigated. Conversions and selectivities were determined by means of GC analyses of the samples taken and are based on the molar amounts in the liquid phase calculated from the peak areas by means of the experimentally determined calibration factors (Table 10).

### Catalyst Screening

The 6 Ru catalysts and 2 Pt catalysts shown in Table 11 were tested in 100 ml of an aqueous solution of furfuryl alcohol at 200° C. and a hydrogen pressure of 100 bar in a batch reactor. Here, 0.5 g of catalyst and the additives indicated were used in each case. The reaction was stopped as soon as complete conversion of furfuryl alcohol had been achieved. Table 11 summarizes the reaction times for complete conversion and the selectivities to the most important reaction products for the 8 catalysts examined.

It can be seen that all Al₂O₃-supported ruthenium catalysts shown in Table 11 reacted with a surprisingly high selectivity to the product 1,2-pentanediol. The selectivity under the conditions selected was in each case higher than, for instance, that described by Zhang et al. for Ru/AlMgO_{4 [}49]. The best selectivity to 1,2-pentanediol was given by 5% Ru/Al₂O₃ (A11) at 32%. As can be seen from Table 12, a high selectivity was also observed without addition of Na₂CO₃. Thus, the ruthenium catalysts generally achieved even better selectivities than the platinum catalysts. In addition, high yields of THFFOH were also observed. These could be achieved at complete conversion of the starting material furfuryl alcohol and thus represent a surprisingly advantageous result compared to the results previously reported in [49].

It was also surprising that in the case of carbon-supported ruthenium catalysts, the selectivity to 1,2-pentanediol was 20 or 21%, and therefore a hundred-fold higher than for the comparable catalyst described by Zhang et al. [49].

Even with 1% Ru/glass (FS384), a selectivity of 14% was observed. In addition, the ruthenium catalysts displayed a higher activity, with 1% Ru/glass also being the exception here. For all catalysts tested, the formation of 1,2-pentanediol (1,2-PD) was strongly favoured over that of 1,5-pentanediol (1,5-PD). The main product was tetrahydrofurfuryl alcohol (THFFOH) which, depending on the catalyst used, was formed in a 1.5- to 3-fold molar ratio relative to 1,2-pentanediol. In addition, 1-pentanol (1-POH), 1,2-butanediol (1,2-BD), 1,4-pentanediol (1,4-PD), cyclopentanone (CpO), cyclopentanol (CpOH), 2-methyltetrahydrofuran (2-MTHF), 1-butanol (1-BOH), presumably 1,5-hexanediol (1,5-HD) and further unknown compounds occurred as by-products.

TABLE 11
Reaction times and selectivities at complete conversion of furfuryl alcohol in aqueous solution.

| | | S | S | S | S | S |
|---|---|---|---|---|---|---|
| | t | (1,2-PD) | (1,5-PD) | (THFFOH) | (1,2-BD) | (CpOH) |
| Catalyst | [min] | [%] | [%] | [%] | [%] | [%] |
| 5% | 60 | 32 | 1 | 59 | 2 | 1 |
| Ru/Al₂O₃ | | | | | | |
| (Al1) ^{a,c} | | | | | | |
| 5% Ru/C | 45 | 21 | 1 | 60 | 10 | 0 |
| (Al2) ^{b} | | | | | | |
| 5% Ru/C | 45 | 20 | 1 | 49 | 7 | 0 |
| (Al3) ^{b} | | | | | | |
| 1% | 40 | 26 | 1 | 60 | 1 | 1 |
| Ru/Al₂O₃ | | | | | | |
| (RR506) ^{b,c} | | | | | | |
| 1% | 60 | 30 | 2 | 61 | 2 | 1 |
| Ru/Al₂O₃ | | | | | | |
| (Y40753-BS) ^{b,c} | | | | | | |
| 10% | 200 | 14 | 1 | 32 | 1 | 10 |
| Ru/glass | | | | | | |
| (FS384) ^{a,c} | | | | | | |
| Pt/PANI | 240 | 14 | 2 | 22 | 0 | 3 |
| (Pt-JA-024) ^{b,d} | | | | | | |
| 10% Pt/C | 180 | 20 | 8 | 34 | 0 | 2 |
| (Pt-JA-026) ^{b,c} | | | | | | |

Reaction conditions: m (Cat) = 0.5 g, T = 200° C., p (H₂) = 100 bar, stirring speed = 1000 rpm, ^{a} c (FFOH) = 40 g/100 ml, ^{b} c (FFOH) = 7.46 g/100 ml, ^{c} 100 mg of Na₂CO₃, ^{d} 300 μ1 total of Na₂CO₃ solution

An attempt was made to construct a comprehensive reaction scheme (scheme 3) which shows the formation of the reaction products identified. For this purpose, a comparison was made with information in the literature and experiments were carried out to confirm individual reaction paths.

A reaction scheme (Scheme 1) for the reaction of furfural over supported platinum catalysts was worked out by Xu et al. [23] and this agrees with the results of the present study for supported platinum and ruthenium catalysts and explains the formation of many of the products detected. According to this scheme, furfuryl alcohol reacts via the following reaction paths:

1. The cleavage of a C—O bond of the furan ring leads to formation of 1,2-pentanediol (6) or 1,5-pentanediol (8). 1,2-Pentanediol can isomerize to 1,4-pentanediol (7).
2. Elimination of the hydroxyl group forms 2-methylfuran (1). This can react by hydrogenation of the furan ring to form 2-methyltetrahydrofuran 2 or by hydrogenolysis of the C-0 bond to form 1-pentanol (4).
3. Hydrogenation of the furan ring forms tetrahydrofurfuryl alcohol (5).

The hydrogenolysis of tetrahydrofurfuryl alcohol to 1,2- or 1,5-pentanediol was negligible under the reaction conditions used by Xu et al., viz. p(H₂)=15 bar and T=130° C. According to Tomishige et al., only a 5% conversion of THFFOH was achieved when using Ru/C at T=120° C. and p(H₂)=80 bar even after a reaction time of 4 hours. [33] However, since this study was carried out using significantly higher temperatures and hydrogen pressures, an experiment using THFFOH as starting material was carried out in order to discover whether the hydrogenolysis of THFFOH is also negligible under more severe reaction conditions. For this purpose, the most active catalyst, viz. 5% Ru/Al₂O₃ (A11), was used at a hydrogen pressure of 100 bar and a reaction temperature of 240° C. It was found that only 24% of the THFFOH had reacted after 15 minutes. 1-BOH was formed as main product with a selectivity of 36%. 1,2-Pentanediol and 1,5-pentanediol were formed in only small amounts with selectivities of 6 and 3%, respectively. It may be concluded from this that 1,2- and 1,5-pentanediol are formed virtually exclusively by hydrogenolysis of FFOH, while the hydrogenolysis of THFFOH plays only a subordinate role. The formation of 1-pentanol from 2-methylfuran proceeds, according to Xu et al., in a manner analogous to the formation of 1,2-pentanediol from FFOH by cleavage of the furan ring at the same C—O bond. The formation of 1-butanol (10) presumably proceeds via furan (9) as intermediate. It is known from the literature that furan can be formed by decarboxylation of furfuryl alcohol with elimination of hydrogen and carbon monoxide at high temperatures, for example using copper catalysts. [45] In order to check whether the formation of 1-butanol can actually be explained by hydrogenolysis of furan, furan was reacted in aqueous solution over 5% Ru/Al₂O₃ (A11) at 175° C. and p(H₂)=100 bar. After a reaction time of 30 minutes, 1-butanol was formed with a selectivity of 15% at 100% conversion. The main product was tetrahydrofuran (THF) with a selectivity of 54%. In order to establish whether this is an intermediate, THF was reacted in aqueous solution over the same catalyst at 240° C. and p(H₂)=100 bar. After 15 minutes, only a low conversion of 14% was observed and the yield of 1-butanol was 3%. The observation that THF is relatively stable even at 240° C., while furan reacts even at 175° C. to give five times the yield of 1-butanol, leads to the conclusion that 1-butanol is formed mainly by hydrogenolysis of furan. This is consistent with the observed trend that the saturated rings (THFFOH, 2-MTHF, THF) are comparatively unreactive in respect of cleavage of the C-0 bond over the supported Ru and Pt catalysts used.

Cyclopentanone (13) and cyclopentanol (14) occurred as by-products with selectivities of up to 17 and 10%, respectively. The conversion of furfural in aqueous solution over Ru and Pt catalysts into these two products under similar reaction conditions has been described. [46] Hronec and Fulajtarova obtained a product mixture in which cyclopentanone occurred as main product (Y=57.33%) and cyclopentanol occurred as by-product (Y=9.50%) using Ru/C at a reaction temperature of 175° C. and a hydrogen pressure of 80 bar. The presence of water plays a critical role in this reaction. This is presumably attributable to the fact that isomerization of furfuryl alcohol to 4-hydroxy-2-cyclopentenone (11) takes place as intermediate step. It is known from the literature that this reaction is observed when heating an aqueous solution of furfuryl alcohol at 200° C. without use of a catalyst. [47] To check this, an experiment was carried out without catalyst under conventional reaction conditions of 200° C. and a hydrogen pressure of 100 bar. Here, the products 4-hydroxy-2-cyclopentenone 11, 2-cyclopentenone 12 and cyclopentanone 13 were detected by GC-MS after a reaction time of 120 minutes. This observation makes it obvious to presume that furfuryl alcohol is firstly isomerized to 4-hydroxy-2-cyclopentenone and subsequently successively reduced to cyclopentenone and cyclopentanone under these conditions. In the presence of a hydrogenation catalyst such as Ru or Pt, the reduction of cyclopentanone to cyclopentanol finally takes place. Since this reaction path has an isomerization which is independent of the catalyst used as first step, it should become increasingly important, the lower the activity of the catalyst. In actual fact, Pt/PANI (Pt-JA-024), viz. the catalyst having the lowest activity, had, at 20%, the greatest overall selectivity to cyclopentanone (S=17%) and cyclopentanol (S=3%).

It was frequently observed that the total molar amount of all products detected did not agree with the molar amount of the starting material used. In order to take account of this fact, the balance Σ was introduced. It is calculated by dividing the sum of the molar amounts of all materials detected (including starting material) in the sample taken by the molar amount of the starting material used. Especially at long reaction times or high temperatures, values calculated for Σ were frequently significantly below 100%. It may be concluded from this that reaction products which cannot be detected by the gas-chromatographic analysis employed are formed. These are presumably polyfurfuryl alcohol which is formed in aqueous solution from furfuryl alcohol. [48] Both the polymerization of furfuryl alcohol and the isomerization to 4-hydroxy-2-cyclopentenone are promoted by acidic conditions. This is a problem since a decrease in the pH was observed during the reaction in all experiments, in particular at long reaction times. Thus, in the blank experiment without catalyst, the pH decreased from 7 to 2.4 after a reaction time of 120 minutes. Polymerization becomes increasingly important, the less active the catalyst used, because it occurs without a hydrogenation catalyst.

The formation of 1,2-butanediol cannot be explained by the reaction scheme constructed. A further compound which in some experiments occurred in small yields was identified as 1,5-hexanediol by GC-MS. However, it is questionable whether this assignment is correct, since no mechanism for introduction of the sixth carbon atom under these conditions has hitherto been known.

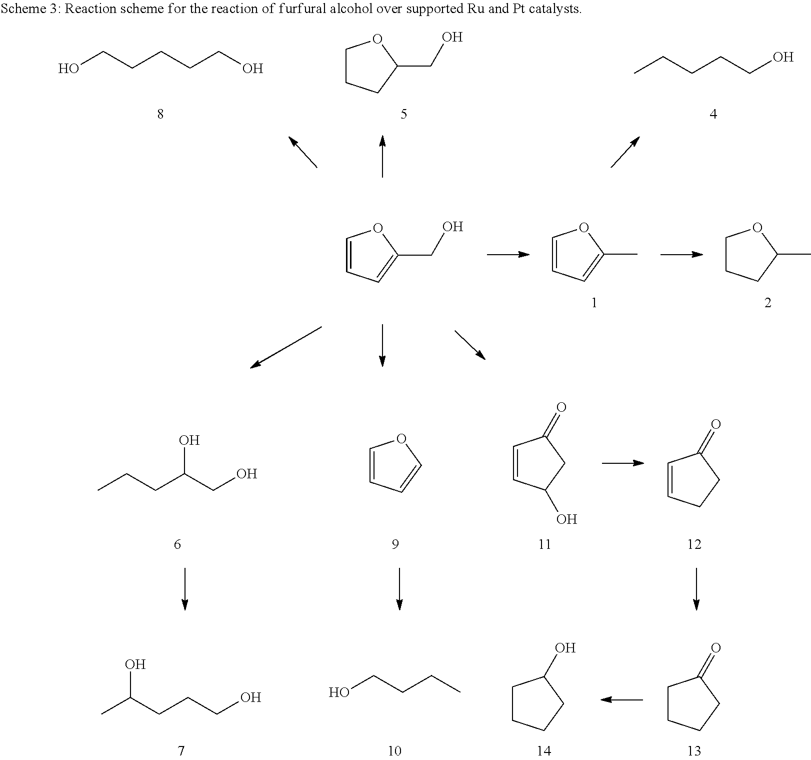

### 2. Examples 1-3

### 3*: Preparation of 1,2-pentanediol using an Ru/Al₇O₃ catalyst (according to the invention

The reaction was carried out in a stirring autoclave (Parr Instruments) made of stainless steel and having a capacity of 300 ml. A 500 ml gas tank was connected to the reactor via a pressure reducer and could be supplied via a gas feed line system both with argon (≦80 bar) and with hydrogen (≦200 bar). An offgas valve served to depressurize the reactor after the end of the experiment. A gas introduction stirrer with a maximum stirring speed of 1600 rpm was used for mixing. A thermostat-controlled heating jacket by means of which temperatures up to 280° C. could be set served for heating the reaction mixture. Samples could be taken via an offtake tap during the reaction. Furthermore, a supply tank to which hydrogen and argon could likewise be introduced from the gas tank was connected to the reactor for the introduction of the starting material. The pressure was measured by means of a manometer and the temperature in the gas and liquid phases was measured separately by means of thermocouples. The courses of pressure and temperature were stored digitally and controlled.

86.8 ml of water and 0.5 g of the 5% Ru/Al₂O₃ catalyst A11 were placed in the autoclave. The reaction apparatus was closed and a stirring speed of 1000 rpm was set. The reactor was flushed three times with 10 bar of argon and once with 10 bar of hydrogen. The hydrogen pressure was subsequently set so that the expected pressure on reaching the reaction temperature would be 10-20 bar below the reaction pressure of 100 bar. The contents of the autoclave were heated to the reaction temperature over a period of 30-60 minutes. The reaction temperature was 150° C. in the case of Example 1, 200° C. in the case of Example 2, 240° C. in the case of Example 3 and 260° C. in the case of Example 3*. As soon as this had been reached, 7.46 g of furfuryl alcohol in 6.6 ml of water were introduced quickly via the supply tank. Further hydrogen was continually supplied via the gas tank so as to keep the reaction pressure constant. For sampling, the offtake line was flushed beforehand with 1-2 ml of reaction mixture and 0.5-1 ml of sample was then taken. When the hydrogen pressure in the gas tank no longer decreased, the heating jacket was removed and the reactor was cooled in air to room temperature. After the reaction mixture had been taken off, the reactor was thoroughly cleaned and baked at 150° C. under 30 bar of argon for 60 minutes.

The product mixture was analysed and the following selectivities were found at the various temperatures (see Table 12).

TABLE 12
Selectivities at complete conversion of furfuryl alcohol in
aqueous solution at various temperatures using 5% Ru/Al₂O₃.

| | | | | | S | | |
|---|---|---|---|---|---|---|---|
| | | | S | S | (1,2-PD)/ | S | |
| | T | t | (1,2-PD) | (THFFOH) | S | (CpOH) | Σ |
| Example | [° C.] | [min] | [%] | [%] | (THFFOH) | [%] | [%] |
| 1 | 150 | 60 | 14 | 73 | 0.19 | 0 | 88 |
| 2 | 200 | 30 | 26 | 53 | 0.49 | 0 | 90 |
| 3 | 240 | 15 | 27 | 25 | 1.08 | 4 | 74 |
| 3* | 260 | 25 | 13 | 9 | 1.44 | 14 | 54 |

Reaction conditions: m (Cat) = 0.5 g, p (H₂) = 100 bar, stirring speed = 1000 rpm, c (FFOH) = 7.46 g/100 ml, * 300 mg of Na₂CO₃.

As can be seen from the table, the ratio of 1,2-pentanediol to 1,5-tetrahydrofurfuryl alcohol is surprisingly 0.19 even at a temperature of 150° C. and thus above the ratio previously described. Even more surprising was the fact that complete conversion was achieved after not more than 60 minutes, with the selectivity to 1,2-pentanediol being 14% even at 150° C., and even 26 and 27% at 200° C. and 240° C., respectively. This optimal temperature accordingly appeared to be 200-240° C. under these reaction conditions.

### Examples 4-8

### According to the Invention

It was surprisingly determined that the selectivity could be increased further when the hydrogenolysis was carried out with addition of small amounts of saturated Na₂CO₃ solution or solid Na₂CO₃.

Specifically, 86.8 ml of water, 0.5 g of the 5% Ru/Al₂O₃ catalyst A11 and the amount indicated in Table 13 of Na₂CO₃ (0 mg, 10 mg, 30 mg, 60 mg, 300 mg) were placed in the autoclave. The reaction apparatus was closed and a stirring speed of 1000 rpm was set. The reactor was flushed three times with 10 bar of argon and once with 10 bar of hydrogen. The hydrogen pressure was subsequently set so that the pressure expected on reaching the reaction temperature would be 10-20 bar below the reaction pressure of 100 bar. The contents of the reactor were heated to the reaction temperature over a period of 30-60 minutes. The reaction temperature was 240° C. As soon as this had been reached, 7.46 of furfuryl alcohol in 6.6 ml of water were quickly introduced via the supply tank. Further hydrogen was continually introduced via the gas tank so as to keep the reaction pressure constant. For sampling, the offtake line was flushed beforehand with 1-2 ml of reaction mixture and 0.5-1 ml of sample was then taken. When the hydrogen pressure in the gas tank no longer decreased, which was the case after 15 minutes for Examples 4-7, and was the case after 25 minutes for Example 8, the heating jacket was removed and the reactor was cooled in air to room temperature. After taking off the reaction mixture, the reactor was thoroughly cleaned and baked at 150° C. under 30 bar of argon for 60 minutes.

The product mixture was analysed and the following selectivities at various temperatures were found:

TABLE 13
Selectivities at complete conversion of furfuryl alcohol in
aqueous solution with addition of various amounts of saturated
Na₂CO₃ solution when using 5% Ru/Al₂O₃ (Al1).

| | m(Na₂CO₃) | pH (after | S(1,2-PD) | S(CpOH) | |
|---|---|---|---|---|---|
| Example | [mg] | reaction) | [%] | [%] | Σ [%] |
| 4 | 0 | 5.3 | 27 | 4 | 74 |
| 5 | 10 | 6.2 | 32 | 7 | 87 |
| 6 | 30 | 7.6 | 35 | 5 | 91 |
| 7 | 60 | 10.2 | 30 | 4 | 80 |
| 8 | 300 | 10.5 | 25 | 3 | 67 |

Reaction conditions: m(Cat) = 0.5 g, T = 240° C., p(H₂) = 100 bar, stirring speed = 1000 rpm, c(FFOH) = 7.46 g/100 ml, t = 15 minutes or in the case of Example 8 25 min.

As can be seen from the table, the selectivity to 1,2-pentanediol firstly increased with the amount of base used. When 150 ml of saturated Na₂CO₃ solution (corresponding to 30 mg of Na₂CO₃) was added, the selectivity was 35% and thus 8% higher than without addition of base. The pH measured in the reaction output after the reaction was slightly basic at 7.6. When 300 μl of Na₂CO₃ solution (corresponding to 60 mg of Na₂CO₃) are used, the selectivity to 1,2-pentanediol decreases again. When 300 mg of Na₂CO₃ in solid form are used, the selectivity is 25% and therefore lower than when no base was added. This pH effect was completely surprising. In particular, it was surprising in the light of previously reported results that the selectivity displayed such a significant increase in a particular pH range [47], [48].

### Examples 9 and 10

To confirm this pH effect, a further corresponding experiment in which sodium acetate was used as base was carried out (Examples 9 and 10). Specifically, 64.4 ml of water, 1.0 g of the 5% Ru/Al₂O₃ catalyst A11 and 100 mg of Na₂CO₃ (Example 9) or 5 g of sodium acetate (Example 10) were placed in the autoclave. The reaction apparatus was closed and a stirring speed of 1000 rpm was set. The reactor was flushed three times with 10 bar of argon and once with 10 bar of hydrogen. The hydrogen pressure was subsequently set so that the pressure expected on reaching the reaction temperature would be 10-20 bar below the reaction pressure of 100 bar. The contents of the reactor were heated to the reaction temperature over a period of 30-60 minutes. The reaction temperature was 200° C. As soon as this had been reached, 40 g of furfuryl alcohol were quickly introduced via the supply tank. Further hydrogen was continually introduced via the gas tank so as to keep the reaction pressure constant. For sampling, the offtake line was flushed beforehand with 1-2 ml of reaction mixture and 0.5-1 ml of sample was then taken. When the hydrogen pressure in the gas tank no longer decreased, which was the case after 30 minutes, the heating jacket was removed and the reactor was cooled in air to room temperature. After the reaction mixture had been taken off, the reactor was thoroughly cleaned and baked at 150° C. under 30 bar of argon for 60 minutes.

The product mixture was analysed and the following selectivities at various temperatures were found (Table 14).

TABLE 14
Selectivities at complete conversion of furfuryl alcohol in aqueous
solution with addition of various bases using 5% Ru/Al₂O₃ (Al1).

| | | pH (after | S(1,2-PD) | S(CpOH) | |
|---|---|---|---|---|---|
| Example | Base | reaction) | [%] | [%] | Σ [%] |
| 9 | 100 mg of | 7.9 | 34 | 1 | ≧100 |
| | Na₂CO₃ | | | | |
| 10 | 5 g of NaOAc | 7.0 | 30 | 1 | ≧100 |

Reaction conditions: m(Cat) = 1.0 g, T = 200° C., p(H2) = 100 bar, stirring speed = 1000 rpm, c(FFOH) = 40.0 g/100 ml, t = 30 minutes (Example 9) or 25 min (Example 10).

Since sodium acetate is a significantly weaker base, large amounts would have to be used to achieve a neutral pH after the reaction. Table 14 shows a comparison of the experimental results obtained when the two bases were used under reaction conditions of T=200° C. and p(H₂)=100 bar. When using 100 mg of Na₂CO₃, a selectivity of 34% was achieved; the selectivity of 30% achieved using 5 g of sodium acetate may be attributed to the formation of unknown by-products which reduce the yield of 1,2-pentanediol. Nevertheless, the selectivity of 30% observed when using sodium acetate was higher than that in the blank test (Example 4 or Example 8).

It was accordingly shown that the surprising positive effect could also be achieved when using bases other than just Na₂CO₃ and was a pH effect.

### Examples 11 and 12

To examine the influence of the starting material concentration on the selectivity distribution, the hydrogenolysis was carried out using two different concentrations of furfuryl alcohol in water at 200° C. under a hydrogen pressure of 100 bar to complete conversion of the starting material. Specifically, in Example 11, 75.0 ml of water, 0.5 g of the 5% Ru/Al₂O₃ catalyst A11 and 100 mg of Na₂CO₃ were placed in the autoclave. In Example 12, 64.4 ml of water, 0.5 g of the Ru/Al₂O₃ catalyst and 100 mg of Na₂CO₃ were placed in the autoclave. The reaction apparatus was closed and a stirring speed of 1000 rpm was set. The reactor was flushed three times with 10 bar of argon and once with 10 bar of hydrogen. The hydrogen pressure was subsequently set so that the pressure expected on reaching the reaction temperature would be 10-20 bar below the reaction pressure of 100 bar. The contents of the reactor were heated to the reaction temperature over a period of 30-60 minutes. The reaction temperature was 200° C. As soon as this had been reached, 20.0 g of furfuryl alcohol in 7.0 ml of water, in the case of Example 11, and 40 g of furfuryl alcohol in the case of Example 12 were quickly introduced via the supply tank. Further hydrogen was continually introduced via the gas tank so as to keep the reaction pressure constant. For sampling, the offtake line was flushed beforehand with 1-2 ml of reaction mixture and 0.5-1 ml of sample was then taken. When the hydrogen pressure in the gas tank no longer decreased, which was the case after 25 minutes for Example 11, and was the case after 60 minutes for Example 12, the heating jacket was removed and the reactor was cooled in air to room temperature. After taking off the reaction mixture, the reactor was thoroughly cleaned and baked at 150° C. under 30 bar of argon for 60 minutes.

The product mixture was analysed and the following selectivities at the various temperatures were found:

The experimental results are listed in Table 15. The differences in the product distribution at concentrations of 20 and 40 g of FFOH in 100 ml of reaction solution were extremely small. The starting material concentration obviously therefore has no appreciable effect on the product selectivities within this concentration range. Since a high starting material concentration is desirable for industrial applications, further experiments using this catalyst were carried out at a concentration of 40 g of FFOH in 100 ml of reaction solution.

TABLE 15
Selectivities at complete conversion of furfuryl alcohol
in aqueous solution using 5% Ru/Al₂O₃ (Al1) with variation
of the starting material concentration.

| | c (FFOH) | S(1,2-PD) | S(THFFOH) | Σ |
|---|---|---|---|---|
| Example | [g/100 ml] | [%] | [%] | [%] |
| 11 | 20 ^{a} | 32 | 60 | ≧100 |
| 12 | 40 ^{b} | 32 | 57 | ≧100 |

Reaction conditions: m(Cat) = 0.5 g, T = 200° C., p(H₂) = 100 bar, stirring speed = 1000 rpm, 100 mg of Na₂CO₃,
^{a} t = 25 minutes,
^{b} t = 60 minutes.

### Examples 13 to 15

To examine the influence of the amount of catalyst on the selectivity distribution, the hydrogenolysis was carried out using different amounts of 5% Ru/Al₂O₃ catalyst A11 at 200° C. under a hydrogen pressure of 100 bar to complete conversion of the starting material. Specifically, 64.4 ml of water, 100 mg of Na₂CO₃ and 0.25 g (Example 13), 0.5 g (Example 14) or 1.0 g (Example 15) of the 5% Ru/Al₂O₃ catalyst were placed in the autoclave. The reaction apparatus was closed and a stirring speed of 1000 rpm was set. The reactor was flushed three times with 10 bar of argon and once with 10 bar of hydrogen. The hydrogen pressure was subsequently set so that the pressure expected on reaching the reaction temperature would be 10-20 bar below the reaction pressure of 100 bar. The contents of the reactor were heated to the reaction temperature over a period of 30-60 minutes. The reaction temperature was 200° C. As soon as this had been reached, 40 g of furfuryl alcohol were quickly introduced via the supply tank. Further hydrogen was continually introduced via the gas tank so as to keep the reaction pressure constant. For sampling, the offtake line was flushed beforehand with 1-2 ml of reaction mixture and 0.5-1 ml of sample was then taken. When the hydrogen pressure in the gas tank no longer decreased, which was the case after 105 minutes for Example 13, after 60 minutes for Example 13 and was the case after 30 minutes for Example 15, the heating jacket was removed and the reactor was cooled in air to room temperature. After taking off the reaction mixture, the reactor was thoroughly cleaned and baked at 150° C. under 30 bar of argon for 60 minutes.

The product mixture was analysed and the following selectivities at the various temperatures were found:

The experimental results are summarized in Table 16.

TABLE 16
Selectivities at complete conversion of furfuryl
alcohol in aqueous solution using
5% Ru/Al₂O₃ (Al1) with variation of the mass of catalyst.

| | m | | S | S | S | |
|---|---|---|---|---|---|---|
| | (Cat) | t | (1,2-PD) | (THFFOH) | (CpOH) | Σ |
| Example | [g] | [min] | [%] | [%] | [%] | [%] |
| 13 | 0.25 | 105 | 23 | 46 | 3 | 79 |
| 14 | 0.5 | 60 | 32 | 57 | 1 | ≧100 |
| 15 | 1.0 | 30 | 34 | 56 | 0 | ≧100 |

Reaction conditions: T = 200° C., p (H₂) = 100 bar, stirring speed = 1000 rpm, c (FFOH) = 40 g/100 ml, 100 mg of Na₂CO₃.

A reduction in the mass of catalyst led to lower selectivities to 1,2-pentanediol. While the selectivity was 34% when using 1 g of catalyst, it was only 23% when using 0.25 g of catalyst. Further trends on reducing the mass of catalyst were increased formation of cyclopentanol and a decrease in the balance E to below 100% when using 0.25 g of catalyst, which indicates the formation of polymerization products. These results therefore surprisingly show that an amount of catalyst of greater than 0.25 g, preferably greater than 0.5 g, particularly preferably from 0.5 g to 1.0 g, of 5% Ru/Al₂O₃ leads to the advantageous selectivity to 1,2-pentanediol.

### Examples 16 to 18

To examine the influence of the hydrogen pressure on the product distribution in the hydrogenolysis of furfuryl alcohol, the reaction was carried out at 200° C. using pressures in the range from 50 to 150 bar.

Specifically, 64.4 ml of water, 100 mg of Na₂CO₃ and 1.0 g of the 5% Ru/Al₂O₃ catalyst A11 were placed in the autoclave. The reaction apparatus was closed and a stirring speed of 1000 rpm was set. The reactor was flushed three times with 10 bar of argon and once with 10 bar of hydrogen. The hydrogen pressure was subsequently set so that the pressure expected on reaching the reaction temperature would be 10-20 bar below the respective reaction pressure of 50 bar, 100 bar or 150 bar. The contents of the reactor were heated to the reaction temperature over a period of 30-60 minutes. The reaction temperature was 200° C. As soon as this had been reached, 40 g of furfuryl alcohol were quickly introduced via the supply tank. Further hydrogen was continually introduced via the gas tank so as to keep the reaction pressure constant. For sampling, the offtake line was flushed beforehand with 1-2 ml of reaction mixture and 0.5-1 ml of sample was then taken. When the hydrogen pressure in the gas tank no longer decreased, which was the case after 140 minutes for Example 16, after 30 minutes for Example 17 and was the case after 25 minutes for Example 18, the heating jacket was removed and the reactor was cooled in air to room temperature. After taking off the reaction mixture, the reactor was thoroughly cleaned and baked at 150° C. under 30 bar of argon for 60 minutes.

The product mixture was analysed and the following selectivities at the various temperatures were found: The experimental results are summarized in Table 17.

TABLE 17
Selectivities in the hydrogenolysis of furfuryl
alcohol in aqueous solution using 5%
Ru/Al₂O₃ (Al1) with variation of the hydrogen pressure.

| | | | S | | S | | |
|---|---|---|---|---|---|---|---|
| | p | | (1,2- | S | (1,2- | S | |
| | (H₂) | t | PD) | (THFFOH) | PD)/S | (CpOH) | Σ |
| Example | [bar] | [min] | [%] | [%] | (THFFOH) | [%] | [%] |
| 16 | 50 ^{a} | 140 | 34 | 38 | 0.89 | 6 | ≧100 |
| 17 | 100 ^{a} | 30 | 34 | 56 | 0.61 | 0 | ≧100 |
| 18 | 150 ^{b} | 25 | 31 | 60 | 0.52 | 0 | ≧100 |

Reaction conditions: m (Cat) = 1.0 g, T = 200° C., stirring speed = 1000 rpm, c (FFOH) = 40 g/100 ml, 100 mg of Na₂CO₃, ^{a} X = 100%, ^{b} X = 99%.

It was surprisingly found that the high selectivity to 1,2-pentanediol could be observed over a wide pressure range. Nevertheless, a dependence of the catalyst activity on the hydrogen pressure was observed: while it took 30 minutes for complete conversion of furfuryl alcohol to be reached at p(H₂)=100 bar, at p(H₂)=50 bar the conversion was virtually complete only after 140 minutes. This decrease in activity results, as expected, in an increased selectivity to cyclopentanol. Thus, cyclopentanol was found in a selectivity of 7% at a hydrogen pressure of 50 bar, while it was detected only in the trace range at p(H₂)=150 bar. The selectivity ratio S(1,2-PD)/S(THFFOH) increases with decreasing pressure.

This leads to the selectivity to 1,2-pentanediol at p(H₂)=50 bar being, at 34%, as high as at p(H₂)=100 bar despite the increased formation of cyclopentanol and further by-products.

### Examples 19 to 22

To estimate the influence of the solvent, the reaction was carried out in three different organic solvents at 200° C. and a hydrogen pressure of 100 bar to complete conversion of furfuryl alcohol.

Specifically, 100 mg of Na₂CO₃, 1.0 g of the 5% Ru/Al₂O₃ catalyst A11 and 64.0 ml of ethanol (Example 20), 63.8 ml of tetrahydrofuran (THF; Example 21), 64 ml of dioxane (Example 22) or 64.6 ml of water with 100 mg of Na₂CO₃ (Example 19) were placed in the autoclave. The reaction apparatus was closed and a stirring speed of 1000 rpm was set. The reactor was flushed three times with 10 bar of argon and once with 10 bar of hydrogen. The hydrogen pressure was subsequently set so that the pressure expected on reaching the reaction temperature would be 10-20 bar below the respective reaction pressure of 100 bar. The contents of the reactor were heated to the reaction temperature over a period of 30-60 minutes. The reaction temperature was 200° C. As soon as this had been reached, 40 g of furfuryl alcohol were quickly introduced via the supply tank. Further hydrogen was continually introduced via the gas tank so as to keep the reaction pressure constant. For sampling, the offtake line was flushed beforehand with 1-2 ml of reaction mixture and 0.5-1 ml of sample was then taken. When the hydrogen pressure in the gas tank no longer decreased, which was the case after 30 minutes for Examples 19 and 21, was the case after 60 minutes for Example 20 and was the case after 45 minutes for Example 22, the heating jacket was removed and the reactor was cooled in air to room temperature. After taking off the reaction mixture, the reactor was thoroughly cleaned and baked at 150° C. under 30 bar of argon for 60 minutes.

The product mixture was analysed and the following selectivities at the various temperatures were found. The experimental results are summarized in Table 18.

TABLE 18
Selectivities at complete conversion of furfuryl alcohol using
5% Ru/Al₂O₃ (Al1) with variation of the solvent.

| | | | S | S | S | |
|---|---|---|---|---|---|---|
| | | t | (1,2-PD) | (THFFOH) | (CpOH) | Σ |
| Example | Solvent | [min] | [%] | [%] | [%] | [%] |
| 19 | Water ^{a} | 30 | 34 | 56 | 0 | ≧100 |
| 20 | Ethanol | 60 | 25 | 61 | 0 | 91 |
| 21 | THF | 30 | 25 | 69 | 0 | ≧100 |
| 22 | 1,4- | 45 | 20 | 64 | 0 | 88 |
| | Dioxane | | | | | |

Reaction conditions: m (Cat) = 1.0 g, T = 200° C., p (H₂) = 100 bar, stirring speed = 1000 rpm, c (FFOH) = 40 g/100 ml, ^{a} 100 mg of Na₂CO₃.

Although the selectivities were also surprisingly good when using the organic solvents mentioned, it was found that the highest selectivity could be obtained when using the solvent water.

### Examples 23 and 24

To examine the influence of diffusion, the hydrogenolysis was carried out at 200° C. and a hydrogen pressure of 100 bar to complete conversion of furfuryl alcohol at 2 different stirring speeds.

Specifically, 64.6 ml of water and 1.0 g of the 5% Ru/Al₂O₃ catalyst A11 were placed together with 100 mg of Na₂CO₃ in the autoclave. The reaction apparatus was closed and a stirring speed of 1000 rpm (Example 23) or 1600 rpm (Example 24) was set. The reactor was flushed three times with 10 bar of argon and once with 10 bar of hydrogen. The hydrogen pressure was subsequently set so that the pressure expected on reaching the reactor temperature would be 10-20 bar below the respective reaction pressure of 100 bar. The contents of the reactor were heated to the reaction temperature over a period of 30-60 minutes. The reaction temperature was 200° C. As soon as this had been reached, 40 g of furfuryl alcohol were quickly introduced via the supply tank. Further hydrogen was continually introduced via the gas tank so as to keep the reaction pressure constant. For sampling, the offtake line was flushed beforehand with 1-2 ml of reaction mixture and 0.5-1 ml of sample was then taken. When the hydrogen pressure in the gas tank no longer decreased, which was the case after 30 minutes for Example 23, and was the case after 25 minutes for Example 24, the heating jacket was removed and the reactor was cooled in air to room temperature. After taking off the reaction mixture, the reactor was thoroughly cleaned and baked at 150° C. under 30 bar of argon for 60 minutes.

The product mixture was analysed and the following selectivities at the various temperatures were found. The experimental results are summarized in Table 19.

TABLE 19
Selectivities at complete conversion of furfuryl
alcohol in aqueous solution using 5% Ru/Al₂O₃ (Al1)
with variation of the stirring speed.

| | Stirring | t | S(1,2-PD) | S(THFFOH) | Σ |
|---|---|---|---|---|---|
| Example | speed [rpm] | [min] | [%] | [%] | [%] |
| 23 | 1000 | 30 | 34 | 56 | ≧100 |
| 24 | 1600 | 25 | 28 | 52 | 85 |

Reaction conditions: m(Cat) = 1.0 g, T = 200° C., p(H₂) = 100 bar, c(FFOH) = 40 g/100 ml, 100 mg of Na₂CO₃.

These examples show that a selectivity S(1,2-PD) of 28% was still achieved and the selectivity was surprisingly good when the stirring speed was increased from 1000 to 1600 rpm.

### Examples 25 to 31

To examine the influence of second metals in the Ru/Al₂O₃ catalyst, six different bimetallic [n(Ru):n(Me)] Ru-Me/Al₂O₃ catalysts based on 5% Ru/Al₂O₃ (A11) with the molar ratio [n(Ru):n(Me)] were prepared as described above (Sections 2.13 to 2.15). Sn, Zn, Fe, Cu, Ni and Pt were used as second metals. While Sn was introduced in situ by addition of Sn(II) chloride, the remaining catalysts were prepared by impregnation with a solution of the metal nitrate. The catalysts were in each case calcined at 200° C. for 1 hour and reduced at 250° C. for 1 hour. Specifically, the hydrogenolysis of furfuryl alcohol was carried out as follows: 0.5 g of the respective bimetallic catalyst (Example 25: 5% Ru/Al₂O₃ A11; Example 26: [17:2] Ru—Sn/Al₂O₃; Example 27: [5:2] Ru—Zn/Al₂O₃; Example 28: [5:2] Ru—Fe/Al₂O₃; Example 29: [1:2] Ru—Cu/Al₂O₃; Example 30: [1:2] Ru—Ni/Al₂O₃; Example 31: [1:2] Ru—Pt/Al₂O₃) was placed in the autoclave. The reaction apparatus was closed and a stirring speed of 1000 rpm was set. The reactor was flushed three times with 10 bar of argon and once with 10 bar of hydrogen. The hydrogen pressure was subsequently set so that the pressure expected on reaching the reaction temperature would be 10-20 bar below the respective reaction pressure of 100 bar. The contents of the reactor were heated to the reaction temperature over a period of 30-60 minutes. The reaction temperature was 200° C. As soon as this had been reached, 40 g of furfuryl alcohol were quickly introduced via the supply tank. Further hydrogen was continually introduced via the gas tank so as to keep the reaction pressure constant. For sampling, the offtake line was flushed beforehand with 1-2 ml of reaction mixture and 0.5-1 ml of sample was then taken. When the hydrogen pressure in the gas tank no longer decreased, which was the case after 60 minutes for Examples 25 and 30, was the case after 20 minutes for Example 26, was the case after 90 minutes for Examples 27 and 28, was the case after 15 minutes for Example 29 and was the case after 180 minutes for Example 31, the heating jacket was removed and the reactor was cooled in air to room temperature. After taking off the reaction mixture, the reactor was thoroughly cleaned and baked at 150° C. under 30 bar of argon for 60 minutes. The product mixture was analysed and the following selectivities were found for the various catalysts. The experimental results are summarized in Table 20.

TABLE 20
Conversions and selectivities in the hydrogenolysis of furfuryl
alcohol in aqueous solution using bimetallic Ru-Me/Al₂O₃ catalysts.

| | | | | S | | | |
|---|---|---|---|---|---|---|---|
| | | | X | (1,2- | S | S | |
| | | t | (FFOH) | PD) | (THFFOH) | (CpOH) | Σ |
| Example | Catalyst | [min] | [%] | [%] | [%] | [%] | [%] |
| 25 | 5% | 60 | 100 | 32 | 57 | 1 | ≧100 |
| | Ru/Al₂O₃ | | | | | | |
| | (Al1) | | | | | | |
| 26 | [17: 2] Ru- | 20 | 0 | — | — | — | — |
| | Sn/Al₂O₃ | | | | | | |
| 27 | [5: 2] Ru- | 90 | 100 | 24 | 54 | 1 | 86 |
| | Zn/Al₂O₃ | | | | | | |
| 28 | [5: 2] Ru- | 90 | 100 | 26 | 48 | 1 | 84 |
| | Fe/Al₂O₃ | | | | | | |
| 29 | [1: 2] Ru- | 15 | 0 | — | — | — | — |
| | Cu/Al₂O₃ | | | | | | |
| 30 | [1: 2] Ru- | 60 | 100 | 7 | 70 | 0 | 82 |
| | Ni/Al₂O₃ | | | | | | |
| 31 | [1: 2] Ru- | 180 | 100 | 10 | 37 | 12 | 71 |
| | Pt/Al₂O₃ | | | | | | |

Reaction conditions: stirring speed = 1000 rpm, m (Cat) = 0.5 g, T = 200° C., p (H₂) = 100 bar, c (FFOH) = 40 g/100 ml, 100 mg of Na₂CO₃.

For comparison, the experimental results using the monometallic 5% Ru/Al₂O₃ in Example 25 under identical reaction conditions are entered; this gave a selectivity of 32% to 1,2-pentanediol. The addition of Sn and Cu reduced the activity of the catalyst to such a degree that no hydrogen consumption could be observed and the reaction was stopped after 20 and 15 minutes, respectively. With a reaction time of 180 minutes, Ru—Pt/Al₂O₃ displayed a low activity, which resulted, as expected, in increased formation of cyclopentanol (S=12%) and presumably increased polymerization (I=71%). The selectivity to 1,2-pentanediol was correspondingly low at 10%. Ru—Ni/Al₂O₃ also displayed a low selectivity of 7% to 1,2-pentanediol, while the main product THFFOH was formed with an extremely high selectivity of 70%. This can be explained by nickel being an active and highly selective catalyst and thus functioning so as to hydrogenate FFOH to THFFOH. [23, 26, 27, 28] It could thus also be seen that Ru—Ni/Al₂O₃ as sole bimetallic catalyst did not have a reduced activity compared to 5% Ru/Al₂O₃ (A11). Ru—Zn/Al₂O₃ and Ru—Fe/Al₂O₃ had a moderate selectivity of 24 and 26%, respectively, to 1,2-pentanediol and displayed a similar selectivity ratio S(1,2-PD)/S(THFFOH) of about 1:2 compared to the monometallic catalyst. In summary, it can be said that the addition of a second metal under selected reaction conditions always led to a reduction in the selectivity to 1,2-pentanediol.

### Examples 32 to 37

To be able to estimate the influence of ionic liquids on the selectivity, further experiments were carried out with addition of particular ionic liquids.

It is known from the literature that even the addition of small amounts of an ionic liquid (IL) can drastically alter the selectivity of a heterogeneously catalysed reaction. [43, 44] The IL is immobilized as a thin layer in the catalyst pores, which is why this method is also known as SCILL (solid catalyst with ionic liquid layer). The hydrogenolysis of furfuryl alcohol over 5% Ru/Al₂O₃ with addition of 3 different ILs based on the dicyanamide anion was examined in the multibatch reactor. In addition, an experiment with addition of the salt sodium dicyanamide was carried out for comparison. To avoid decomposition of the ILs, the reaction was carried out at 180° C.

The experiments for comparing the catalyst performance with addition of various ionic liquids were carried out in a multibatch plant. The experimental set-up was as follows: The multibatch plant comprised five reactors made of stainless steel and each having a capacity of 40 ml. Mixing was ensured by means of a five-fold magnetic stirrer. Each reactor was preceded by a 75 ml gas tank which is made of stainless steel and could be supplied with argon or hydrogen at ≦100 bar via the gas feed line system. The individual reactors could be heated independently by means of a thermostat-controlled battery of heating blocks. Control and monitoring of the reaction temperature were computer-controlled.

A suspension composed of 4 g of FFOH, 6.1 ml of water, 100 mg of the 5% Ru/Al₂O₃ catalyst A11 and 2.5 mg of sodium dicyanamide (Example 32) or 5 μl of ionic liquid (Example 33: 1-butyl-3-methylimidazolium dicyanamide; Example 34: N-butyl-3-methylpyridinium dicyanamide; Example 35: 1-butyl-1-methylpyrrolidinium dicyanamide) was placed in the reactor. The reactor was closed and connected to the plant. After flushing three times with 30 bar of argon and once with 50 bar of hydrogen, the reactor was pressurized to 85 bar with hydrogen and heated to 180° C. The pressure temporally reached up to 90 bar during heating and towards the end of the reaction dropped to about 75 bar. The experiment is stopped 100 minutes after the reaction temperature has been reached. For this purpose, the pressure line of the reactor was disconnected from the plant and the reactor was cooled to room temperature in a water bath. The reactor was depressurized to ambient pressure by carefully opening the needle valve and the reaction mixture was taken from the reactor. The experimental results are summarized in Table 21.

TABLE 21
Selectivities at complete conversion of furfuryl alcohol in aqueous
solution using 5% Ru/Al₂O₃ (Al1) with various additives.

| | | S(1,2-PD) | S(THFFOH) | Σ |
|---|---|---|---|---|
| Example | Additive | [%] | [%] | [%] |
| 32 | NaDCA ^{a} | 15 | 65 | 85 |
| 33 | 1-Butyl-3-methyl- | 15 | 80 | ≧100 |
| | imidazolium DCA ^{b} | | | |
| 34 | N-Butyl-3- | 14 | 82 | ≧100 |
| | methylpyridinium DCA ^{b} | | | |
| 35 | 1-Butyl-1-methyl- | 13 | 77 | 92 |
| | pyrrolidinium DCA ^{b} | | | |

Reaction conditions: m(Cat) = 100 mg, T = 180° C., p(H₂)Start = 85 bar, c(FFOH) = 4 g/10 ml, 10 mg of Na₂CO₃,
^{a} 2.5 mg of NaDCA,
^{b} 5 μl of IL.

In all experiments, THFFOH was formed as main product with high selectivities of 65-82%. 1-Butyl-3-methylimidazolium DCA displayed, at 15%, the highest selectivity to 1,2-pentanediol. To be able to make a better comparison with the previous experimental results without ILs, 1-butyl-3-methylimidazolium DCA was once again studied in the batch reactor. For this purpose, the hydrogenolysis of furfuryl alcohol was carried out at 180° C. and a hydrogen pressure of 100 bar over 5% Ru/Al₂O₃ (A11) with addition of 50 μl of 1-butyl-3-methylimidazolium DCA to complete conversion. Here, a selectivity to 1,2-pentanediol of 23% was achieved, with THFFOH being the main product with a selectivity of 60% (Table 22). Compared to the previous experiment carried out without addition of IL at 200° C. under otherwise identical reaction conditions, the selectivity ratio S(1,2-PD)/S(THFFOH) was thus lower. However, this observation may also be explained by the 20° C.-lower reaction temperature since, according to previous observations, a temperature reduction leads to a reduction in S(1,2-PD)/S(THFFOH).

TABLE 22
Selectivities at complete conversion of furfuryl alcohol in aqueous
solution using 5% Ru/Al₂O₃ (Al1) with and without addition of IL.

| | | T | S(1,2-PD) | S(THFFOH) | Σ |
|---|---|---|---|---|---|
| Example | IL | [° C.] | [%] | [%] | [%] |
| 36 | 1-Butyl-3-methyl- | 180 | 23 | 60 | 88 |
| | imidazolium | | | | |
| | DCA ^{a} | | | | |
| 37 | None ^{b} | 200 | 34 | 56 | ≧100 |

Reaction conditions: m(Cat) = 1.0 g, p(H₂) = 100 bar, c(FFOH) = 40 g/100 ml, 100 mg of Na₂CO₃, 50 μl of IL,
^{a} t = 25 min,
^{b} t = 30 min.

### Examples 2/1 to 2/4

### Screening of the Pt(IV)O Catalysts

The hydrogenolysis of furfuryl alcohol was examined in the batch reactor over four different Pt(IV) oxide catalysts in ethanol as solvent. The reaction was carried out by a method based on the literature at room temperature and a hydrogen pressure of 2 bar gauge.
[18] An exception was the catalyst Pt-JA-023. Owing to its low activity, hydrogen pressure and reaction temperature were increased to up to 180° C. and 60 bar during the reaction. The experimental results are summarized in Table 23. With the exception of Pt-JA-023, all catalysts achieved high conversions of over 96% within a reaction time of 2-5 h. A broad product spectrum consisting of 1,2-PD, 1,5-PD, THFFOH, 1-POH, 2-MF, 2-MTHF and also further unknown by-products was obtained. This roughly agrees with the reaction products previously reported. [21] Compared to the supported catalysts (Table 11), a significantly higher ratio of 1,5-PD to 1,2-PD was obtained. In the case of the Pt(IV) oxide catalyst from Heraeus, even more 1,5-PD (23% selectivity) than 1,2-PD was formed. The highest selectivity to 1,2-pentanediol of 25% was achieved by the Pt(IV) oxide catalyst from Sigma Aldrich. It is conspicuous that the calculated balance E is significantly below 100% for all catalysts tested. This can presumably be attributed to production of large amounts of unknown by-products for which no GC calibration data were available.

TABLE 23
Conversions and selectivities in the hydrogenolysis of furfuryl
alcohol in ethanol over Pt (IV) oxide catalysts.

| | | | | p | X | Selec- | |
|---|---|---|---|---|---|---|---|
| Exam- | | t | T | (H₂) | (FFOH) | tivities | Σ |
| ple | Catalyst | [min] | [° C.] | [bar gauge] | [%] | [%] | [%] |
| 2/1 | Pt | 120 | 25-180 | 20-60 | 64 | 1,2-PD: 13 | 94 |
| | (IV) | | | | | 1,5-PD: 11 | |
| | Oxide | | | | | THFFOH: 46 | |
| | Pt-JA- | | | | | 1-POH: 7 | |
| | 023 | | | | | Others: 15 | |
| 2/2 | Pt | 300 | 25-50 | 2 | 99 | 1,2-PD: 11 | 94 |
| | (IV) | | | | | 1,5-PD: 23 | |
| | Oxide | | | | | THFFOH: 53 | |
| | Heraeus | | | | | 1-POH: 2 | |
| | | | | | | Others: 6 | |
| 2/3 | Pt | 120 | 25 | 2 | 100 | 1,2-PD: 19 | 89 |
| | (IV) | | | | | 1,5-PD: 3 | |
| | Oxide | | | | | THFFOH: 24 | |
| | Pt-JA- | | | | | 1-POH: 14 | |
| | 021 | | | | | 2-MTHF: 12 | |
| | | | | | | Others: 16 | |
| 2/4 | Pt | 270 | 25 | 2 | 96 | 1,2-PD: 25 | 93 |
| | (IV) | | | | | 1,5-PD: 3 | |
| | Oxide | | | | | THFFOH: 24 | |
| | Sigma | | | | | 1-POH: 11 | |
| | Aldrich | | | | | 2-MTHF: 6 | |
| | | | | | | Others: 20 | |

Reaction conditions: stirring speed = 1000 rpm, m (Cat) = 0.5 g, c (FFOH) = 10 g/100 ml.

### Examples 2/5 to 2/6

### Variation of the Solvent

Apart from ethanol, acetic acid has also been reported as a solvent for the hydrogenolysis of furfuryl alcohol over Pt(IV) oxide. [22] An experiment in acetic acid using Pt(IV) oxide (Heraeus) was therefore carried out. A large decrease in the hydrogen consumption was observed during the reaction, and the temperature was therefore increased to 75° C. after a reaction time of 1 h. At a conversion of only 38%, the reaction stopped and no further consumption of hydrogen could be observed. The selectivity to 1,2-pentanediol was 6% and thus only about half that when the reaction was carried out in ethanol (Table 24). The ratio of 1,2-PD to 1,5-PD, on the other hand, could be improved from 1:2 to 6:1 in acetic acid. Large amounts of unknown by-products were formed with a total selectivity of about 38%.

TABLE 24
Comparison of the conversions and selectivities in the hydrogenolysis of furfuryl
alcohol over Pt (IV) oxide (Heraeus) in ethanol and acetic acid.

| | | | | p | X | | |
|---|---|---|---|---|---|---|---|
| | | t | T | (H₂) | (FFOH) | Selectivities | Σ |
| Example | Catalyst | [min] | [° C.] | [bar gauge] | [%] | [%] | [%] |
| 2/5 | Pt (IV) oxide ^{a} | 300 | 25-50 | 2 | 99 | 1,2-PD: 11 | 94 |
| | Heraeus | | | | | 1,5-PD: 23 | |
| | | | | | | THFFOH: 53 | |
| | | | | | | 1-POH: 2 | |
| | | | | | | Others: 6 | |
| 2/6 | Pt (IV) oxide ^{b} | 150 | 25-75 | 2 | 38 | 1,2-PD: 6 | 83 |
| | Heraeus | | | | | 1,5-PD: 1 | |
| | | | | | | THFFOH: 7 | |
| | | | | | | 1,2-BD: 1 | |
| | | | | | | 1,5-HD: 1 | |
| | | | | | | 1-POH: 1 | |
| | | | | | | Others: 38 | |

Reaction conditions: stirring speed = 1000 rpm, m (Cat) = 0.5 g, c (FFOH) = 10 g/100 ml, ^{a} solvent: ethanol,
^{b} solvent: acetic acid.

### Examples 2/7 to 2/10

### Influence of Hydrochloric Acid

Previous reports have indicated that when the reaction is carried out in ethanol, small amounts of additives are usually added. While Adams used iron chloride, Nishimura utilized small amounts of dilute hydrochloric acid. [19, 21] To examine the influence of hydrochloric acid on activity and selectivity, experiments were carried out using two different Pt(IV) oxide catalysts in ethanol with addition of in each case 1 ml of 2N hydrochloric acid at room temperature and a hydrogen pressure of 2 bar gauge. The experimental results are listed in Table 25 in comparison with the results achieved without addition of hydrochloric acid under identical reaction conditions. In both cases, the addition of hydrochloric acid led to a reduction in the selectivity to THFFOH. While an improvement in S(1,2-PD) from 19 to 22% occurred when using Pt-JA-021, this deteriorated from 25 to 20% in the case of the catalyst from Sigma Aldrich. At the same time, a drastic increase in the activity was observed in the case of the latter catalyst. Thus, complete conversion of FFOH was achieved after a reaction time of 90 minutes when using hydrochloric acid, while the conversion without addition of hydrochloric acid was 96% after 270 minutes. In the case of Pt-JA-021, on the other hand, the presence of hydrochloric acid had barely any effect on the activity.

TABLE 25
Comparison of the conversions and selectivities in the hydrogenolysis of furfuryl
alcohol over Pt (IV) oxide in ethanol with and without addition of hydrochloric acid.

| | | | V (2N | | | |
|---|---|---|---|---|---|---|
| | | | hydrochloric | X | S | S |
| | | t | acid) | (FFOH) | (1,2-PD) | (THFFOH) |
| Example | Catalyst | [min] | [ml] | [%] | [%] | [%] |
| 2/7 | Pt (IV) oxide | 120 | 0 | 100 | 19 | 24 |
| | (Pt-JA-021) | | | | | |
| 2/8 | Pt (IV) oxide | 130 | 1 | 100 | 22 | 20 |
| | (Pt-JA-021) | | | | | |
| 2/9 | Pt (IV) oxide | 270 | 0 | 96 | 25 | 24 |
| | (Sigma Aldrich) | | | | | |
| 2/10 | Pt (IV) oxide | 90 | 1 | 100 | 20 | 11 |
| | (Sigma Aldrich) | | | | | |

Reaction conditions: stirring speed = 1000 rpm, m (Cat) = 0.5 g, T = 25° C., p (H₂) = 2 bar gauge, c (FFOH) = 10 g/100 ml.

### Examples 2/11 to 2/15

### Influence of Pressure and Temperature

To examine the dependence of the activity and selectivity distribution on the reaction conditions in more detail, experiments using Pt(IV) oxide (Sigma Aldrich), the most selective of the Adams catalysts tested, were carried out with variation of hydrogen pressure and temperature in ethanol.

An increase in the reaction temperature from 25 to 75° C. at a hydrogen pressure of 2 bar gauge led to no appreciable change in S(1,2-PD) (Table 26). While S(THFFOH) decreased from 24 to 17%, other, usually unknown, by-products were formed to an increased extent. The hydrogen consumption decreased extraordinarily quickly during the reaction, and thus no further consumption of hydrogen could be observed at a conversion of only 87%. This observation makes it obvious to presume that deactivation of the catalyst occurred under these conditions.

TABLE 26
Comparison of the conversions and selectivities in the
hydrogenolysis of furfuryl alcohol over Pt(IV) oxide
(Sigma Aldrich) in ethanol at various temperatures.

| | | | X (FFOH) | S(1,2-PD) | S(THFFOH) |
|---|---|---|---|---|---|
| Example | T [° C.] | t [min] | [%] | [%] | [%] |
| 2/11 | 25 | 270 | 96 | 25 | 24 |
| 2/12 | 75 | 270 | 87 | 25 | 17 |

Reaction conditions: stirring speed = 1000 rpm, m(Cat) = 0.5 g, p(H2) = 2 bar gauge, c(FFOH) = 10 g/100 ml.

To examine the influence of the hydrogen pressure, the hydrogenolysis was carried out under a gauge pressure of 5 bar and also under atmospheric pressure at room temperature with addition of 1 ml of 2N hydrochloric acid (Table 27). In contrast to the otherwise usual batch reactor, a glass apparatus was used for carrying out the reaction under atmospheric pressure, with hydrogen being introduced continuously into the reaction solution. A strong dependence of the catalyst activity on the hydrogen pressure was observed. The reaction proceeded so quickly at p(H₂)=5 bar that the temperature increased to up to 50° C. due to the heat of reaction. In addition, the increase in pressure led to an improvement in the selectivity to 1,2-PD from 20 to 23%. When the reaction was carried out under atmospheric pressure, a conversion of only 42% was achieved after a reaction time of 270 minutes, but the selectivity to 1,2-PD was 31%.

TABLE 27
Comparison of the conversions and selectivities in the
hydrogenolysis of furfuryl alcohol over Pt(IV) oxide (Sigma
Aldrich) in ethanol at various hydrogen pressures.

| | p(H₂) [bar | | X (FFOH) | S(1,2-PD) | S(THFFOH) |
|---|---|---|---|---|---|
| Example | gauge] | t [min] | [%] | [%] | [%] |
| 2/13 | 0 | 270 | 42 | 31 | 15 |
| 2/14 | 2 | 90 | 100 | 20 | 11 |
| 2/15 | 5 ^{a} | 20 | 99 | 23 | 11 |

Reaction conditions: stirring speed = 1000 rpm, m(Cat) = 0.5 g, T = 25° C., c(FFOH) = 10 g/100 ml, 1 ml of 2N hydrochloric acid,
^{a} increase in the temperature during the reaction to up to 50° C.

### Summary of the Results

This invention describes studies on the hydrogenolysis of furfuryl alcohol over Ru and Pt catalysts in respect of their selectivity to the desired product 1,2-pentanediol. While Pt(IV) oxide was sufficiently active even at room temperature and a hydrogen pressure of 1-5 bar, more severe reaction conditions of T≧150° C. and p(H2)≧50 bar were required in the case of supported Ru and Pt catalysts.

Four different Pt(IV) oxide catalysts were used. Reaction products obtained in addition to 1,2-pentanediol were 1,5-pentanediol, 1-pentanol, 2-methyltetrahydrofuran, tetrahydrofurfuryl alcohol and large amounts of unidentified substances, with the selectivity distribution being greatly dependent on the catalyst. The most selective of the four Pt(IV) oxide catalysts tested, which had been procured from Sigma Aldrich, achieved a 25% selectivity to 1,2-pentanediol at 96% conversion at room temperature and a hydrogen pressure of 2 bar gauge in ethanol. This corresponds to a yield of 24%. An increase in the reaction temperature led to a decrease in the activity, presumably because of deactivation of the catalyst during the course of the reaction. When the reaction was carried out at room temperature and atmospheric pressure, a selectivity of 31% could be achieved at a conversion of 42%. Addition of small amounts of dilute hydrochloric acid reduced the selectivity to tetrahydrofurfuryl alcohol. The influence of hydrochloric acid on the selectivity to 1,2-pentanediol was dependent on the catalyst. While it decreased from 25 to 20% when Pt(IV) oxide (Sigma Aldrich) was used, it increased from 19 to 22% when Pt(IV) oxide (Pt-JA-021) was used.

In the hydrogenolysis of furfuryl alcohol in water over eight different supported Ru and Pt catalysts (m=0.5 g) at 200° C. and a hydrogen pressure of 100 bar with complete conversion, tetrahydrofurfuryl alcohol was always formed as main product. The best yields of 1,2-pentanediol were given under these reaction conditions by the 5% Ru/Al₂O₃ catalyst A11 at 32%. Further experiments were therefore carried out using this catalyst with variation of the reaction conditions. The most important by-products were generally 1-butanol, cyclopentanol and 1,2-butanol. The formation of 1-butanol is probably attributable to the hydrogenolysis of furan which is formed by decarboxylation of furfuryl alcohol. The formation of cyclopentanol can be explained by isomerization of furfuryl alcohol to 4-hydroxy-2-cyclopentenone with subsequent successive hydrogenation via the intermediates cyclopentenone and cyclopentanone. The mass balance calculated from the gas-chromatographic analysis was frequently below 100%, which indicates polymerization of furfuryl alcohol. Both the polymerization and the isomerization to 4-hydroxy-2-cyclopentenone do not require a catalyst and also proceed without the presence of hydrogen, so that they become prominent especially in the case of a low mass of catalyst and a low hydrogen pressure. An increase in the reaction temperature led to increased polymerization and to increased formation of cyclopentanol. In contrast, the selectivity to tetrahydrofurfuryl alcohol decreased with increasing temperature. It was surprisingly found that control of the pH plays a critical role, especially at high reaction temperatures, and a surprising effect can be achieved in respect of the improved selectivity to 1,2-pentanediol. The inventors presume that the polymerization of furfuryl alcohol and the isomerization to 4-hydroxy-2-cyclopentenone occur to an increased extent under acidic conditions. The addition of small amounts of sodium carbonate enabled the selectivity to 1,2-pentanediol at 240° C. with complete conversion to be increased from 27% to up to 35%. This corresponds to the best selectivity achieved. When larger amounts of base were added, the selectivity to 1,2-pentanediol decreased. In general, a decrease in the pH during the reaction was observed. The selectivity to tetrahydrofurfuryl alcohol could be decreased by reducing the hydrogen pressure. However, owing to the increased formation of cyclopentanol, no improvement in the selectivity to 1,2-pentanediol could be achieved thereby. The selectivities changed only slightly when the starting material concentration was varied. Although a change to organic solvents enabled the formation of cyclopentanol to be prevented, it led to an increased selectivity to tetrahydrofurfuryl alcohol. The selectivity to 1,2-pentanediol when the reaction is carried out in organic solution was 20-25% and thus significantly lower than that in water. S(1,2-PD) at T=200° C. and p(H₂)=100 bar could be improved slightly from 32 to 34% by increasing the mass of catalyst, while a reduction in the mass of catalyst led to a reduction in selectivity and increased polymerization. The addition of small amounts of ionic liquids using the SCILL concept and the use of bimetallic Ru-Me/Al₂O₃ catalysts gave no improvement in the selectivity to 1,2-pentanediol under the conditions set.

### LITERATURE INDEX

[1] http://www.mnforsustain.org/oil_peaking_of_world_oil_production_study_hirsch.htm
[2] http://www.fnr.de
[3] http://www.eere.energy.gov
[4] http://www.suschem.org
[5] http://www.cefic.org
[6] A. Corma, S. Iborra, A. Velty, Chem. Rev. 2007, 107, 241-2502
[7] H. E. Hoydonckx et al., Ullmann's Encyclopedia of Industrial Chemistry WILEY-VCH, DOI: 10.1002/14356007.a12_{—}119.pub2
[8] P. Gallezot, Chem. Soc. Rev. 2012, 41, 1538-1558
[9] P. Anastas, N. Eghbali, Chem. Soc. Rev. 2010, 39, 301-312
[10] P. Anastas, M. Kirchhoff, T. Williamson, Applied Catalysis A: General 2001, 221, 3-13
[11] J. W. Döbereiner, Berichte der deutschen chemischen Gesellschaft 1832, 3, 141-146
[12] B. A. Tokay, Ullmann's Encyclopedia of Industrial Chemistry WILEY-VCH, DOI: 10.1002/14356007.a04_{—}099
[13] W. Lazier, 1931, U.S. Pat. No. 2,077,422
[14] H. Adkins, R. Conner, 1932, U.S. Pat. No. 2,094,975
[15] . S. Goldstein, W. A. Dreher, Industrial and Engineering Chemistry 1960, 52, 57-58
[16] Degussa, D E 2937840, 1981 (G. Kabisch, H. Malitius, S. Raupach, R. Triibe, H. Wittmann)
[17] R. Kadyrov et al., Angew. Chem. Int. Ed. 2009, 48, 7556-7559
[18] L. B. Hunt, Platinum Metals Rev. 1962, 6, 150-152
[19] W. E. Kaufmann, R. Adams, J. Am. Chem. Soc. 1923, 45, 3029-3044
[20] S, Nishimura, Handbook of Heterogeneous Catalytic Hydrogenation for Organic Synthesis, John Wiley & Sons (2001)
[21] S, Nishimura, Bulletin of the Chemical Society of Japan 1961, 34, 32-36
[22] H. A. Smith, J. F. Fuzek, J. Am. Chem. Soc. 1949, 71, 415-419
[23] W. Xu et al., Chem. Commun. 2011, 47, 3924-3926
[24] H. Adkins, R. Connor, J. Am. Chem. Soc. 1931, 53, 1091-1095
[25] D. G. Manly, A. P. Dunlop, J. Org. Chem. 1958, 23, 1093-1095
[26] N. Merat, C. Godawa, A. Gaset, J. Chem. Tech. Biotechnol. 1990, 48, 145-159
[27] Y. Song et al., Front. Chem. Eng. China 2007, 1, 151-154
[28] B. H. Wojcik, Ind. Eng. Chem. 1948, 40, 210-216
[29] G. J. Leuck, J. Pokorny, F. N. Peters 1934, U.S. Pat. No. 2,097,493
[30] F. A. Khan, A. Vallat, G. Süss-Fink, Catal. Commun. 2011, 12, 4128-1431
[31] M. H. Tike, V. V. Mahajani, Ind. Eng. Chem. Res. 2007, 46, 3275-3282
[32] P. D. Seemuth, 1984, U.S. Pat. No. 4,459,419
[33] K. Tomishige et al., Chem. Comm 2009, 15, 2035-2037
[34] K. Tomishige, S. Koso, Y. Nakagawa, Journal of Catalysis 2011, 2, 221-229
[35] A. Suzuki et al., Catal. Sci. Technol. 2011, 1, 1466-1471
[36] P. Walden, Bull Acad. Sci. St. Petersburg 1914, 405-422
[37] J. S. Sandhu, Green Chemistry Letters and Reviews 2011, 4, 289-310
[38] S. E. Fry, N. J. Pienta, J. Am. Chem. Soc. 1985, 107, 6399-6400
[39] J. S. Wilkes et al., J. Org. Chem. 1986, 51, 480-483
[40] A. Jess et al., Chem. Eng. Technol. 2007, 30, 985-994
[41] M. Stark et al., Adv. Mater 2011, 23, 2571-2587
[42] P. Claus et al., J. Phys. Chem. 2010, 114, 10520-10526
[43] P. Claus et al., Chem. Commun. 2008, 4058-4060
[44] P. Claus et al., Chem. Commun. 2009, 11, 716-723
[45] Y.-W. Li et al., Journal of Molecular Catalysis A: Chemical 2006, 246, 18-23
[46] M. Hronec, K. Fulajtarova, Catalysis Communications 2012, 24, 100-104
[47] K. Ulbrich, P. Kreitmeier, O. Reiser, SYNLETT 2010, 13, 2037-2040
[48] T. A. Krishnan, M. Chanda, Die Angewandte Makromolekulare Chemie 1974, 43, 145-156
[48] B. Zhang, Y. Zhang, G. Ding, H. Zheng, Y. Li, Green Chem. 2012, 14, 3402-3409
[50] X. Wang, L. Meng, F. Wu, Y. Jiang, L. Wand, X. Mu, Green Chem. 2012, 14, 758-765
[51] H. Zhao, J, Xing, C. Liu, J. Univ. Petrol., China 2003, 27, 1-19
[52] Y. Hachihama, M. Imoto, K. Kawata, J. Soc. Chem. Ind., Japan 1942, 45, 189-190
[53] D. Papa, E. Schwenk, H. F. Ginsberg, J. Org. Chem. 1951, 16, 253-261
[54] R. Connor, H. Adkins, J. Am. Chem. Soc. 1932, 54, 4678-4690
[55] W. Klaffke, P. Pudlo, D. Springer, J. Thiem, Liebigs Ann Chemie 1991, 509-512
[56] S. R. Vidyarthi, P. C. Nigam, J. K. Gehlawat, J. Sci. Ind. Res. 1983, 42, 268-272
[57] S. Poncet, Speciality Chemicals Magazine 2012, March, 24
[58] M. Chatterjee, H. Kawanami, T. Ishizaka, M. Sato, T. Suzuki, A. Suzuki, Catal. Sci. Tech. 2011, 1, 1466-1471
[59] EP0576828B2
[60] U.S. Pat. No. 4,459,419
[61] EP1767520A1
[62] EP1020473B1

1. A process for preparing 1,2-pentanediol, the process comprising:
reacting furfuryl alcohol with hydrogen in the presence of a catalyst system to obtain a reaction mixture comprising 1,2-pentanediol;
wherein the catalyst system comprises:
(I) ruthenium and
(II) a support comprising aluminum oxide;
wherein the ruthenium is supported on the aluminum oxide.

2. The process according to claim 1, wherein the aluminum oxide is Al₂O₃.

3. The process according to claim 1, wherein a content of the ruthenium is from 0.01 to 30% by weight, based on a total weight of the catalyst system.

4. The process according to claim 1, wherein a BET surface area of the catalyst system is from 50 to 250 m²/g.

5. The process according to claim 1, wherein an average pore volume of the catalyst system is from 0.2 to 0.8 ml/g.

6. The process according to claim 1, wherein a X-ray diffraction pattern of the catalyst system is a pattern comprising the following:

| Pos. | Relative |
|---|---|
| [°2Th.] | Intensity[%] |
| 18.3 | 5-10 |
| 18.8 | 12-17 |
| 20.33 | 6-11 |
| 31.37 | 47-53 |
| 32.68 | 25-32 |
| 32.825 | 80-85 |
| 35.09 | 10-15 |
| 36.758 | 53-59 |
| 38.97 | 27-32 |
| 39.8 | 30-35 |
| 40.63 | 8-13 |
| 43.359 | 14-18 |
| 44.85 | 62-67 |
| 45.47 | 45-50 |
| 46.54 | 25-30 |
| 47.61 | 30-35 |
| 50.77 | 8-13 |
| 51.5 | 7-12 |
| 57.48 | 7-11 |
| 59.97 | 15-20 |
| 62.38 | 8-13 |
| 62.848 | 8-13 |
| 64.05 | 10-16 |
| 65.7 | 10-15 |
| 66.52 | 54-59 |
| 67 | 31-37 |
| 67.4 | 100 |
| 73.5 | 5-10 |

7. The process according to claim 1, wherein a reaction temperature is from 100° C. to 280° C.

8. The process according to claim 1, wherein a reaction pressure is equal to or greater than 10 bar.

9. The process according to claim 1, wherein the reaction is conducted in a solvent selected from the group consisting of water, ethanol, tetrahydrofuran and 1,4-dioxane.

10. The process according to claim 1, wherein a pH of the reaction is from 5.3 to 10.5.

11. The process according to claim 1, wherein the reaction is conducted continuously.

12. The process according to claim 1, wherein the reaction is conducted batchwise.

13. The process according to claim 1, wherein the catalyst system is obtained by a method comprising:
a) producing a mixture (i) comprising a ruthenium salt solution (ii) and an aqueous suspension of aluminum oxide (iii);
b) bringing the mixture (i) to a temperature of from greater than 0° C. to less than 100° C. and a pH of 0 to 14;
c) separating off the catalyst system by filtering the mixture (i) after treatment according to b); and
d) drying the separated catalyst system obtained in c).

14. A process for producing a catalyst system, the process comprising:
a) producing a mixture (i) comprising a ruthenium salt solution (ii) and an aqueous suspension of aluminum oxide (iii);
b) bringing the mixture (i) to a temperature of from greater than 0° C. to less than 100° C. and a pH of 0 to 14;
c) separating off the catalyst system by filtering the mixture (i) after treatment according to b); and
d) drying the separated catalyst system obtained in c).

15. A catalyst system obtained by the process according to claim 14.

16. A process for preparing tetrahydrofurfuryl alcohol, the process comprising:
reacting furfuryl alcohol with hydrogen in the presence of a catalyst system to obtain a reaction mixture comprising tetrahydrofurfuryl alcohol;
wherein the catalyst system comprises:
(I) ruthenium and
(II) a support comprising aluminum oxide;
wherein the ruthenium is supported on the aluminum oxide.

17. The process according to claim 16, wherein the aluminum oxide is Al₂O₃.

18. The process according to claim 16, wherein a content of the ruthenium is from 0.01 to 30% by weight, based on a total weight of the catalyst system.

19. The process according to claim 16, wherein a BET surface area of the catalyst system is from 50 to 250 m²/g.

20. The process according to claim 16, wherein an average pore volume of the catalyst system is from 0.2 to 0.8 ml/g.

21. The process according to claim 16, wherein a X-ray diffraction pattern of the catalyst system is a pattern comprising the following:

| Pos. | Relative |
|---|---|
| [°2Th.] | Intensity [%] |
| 18.3 | 5-10 |
| 18.8 | 12-17 |
| 20.33 | 6-11 |
| 31.37 | 47-53 |
| 32.68 | 25-32 |
| 32.825 | 80-85 |
| 35.09 | 10-15 |
| 36.758 | 53-59 |
| 38.97 | 27-32 |
| 39.8 | 30-35 |
| 40.63 | 8-13 |
| 43.359 | 14-18 |
| 44.85 | 62-67 |
| 45.47 | 45-50 |
| 46.54 | 25-30 |
| 47.61 | 30-35 |
| 50.77 | 8-13 |
| 51.5 | 7-12 |
| 57.48 | 7-11 |
| 59.97 | 15-20 |
| 62.38 | 8-13 |
| 62.848 | 8-13 |
| 64.05 | 10- 16 |
| 65.7 | 10- 15 |
| 66.52 | 54-59 |
| 67 | 31-37 |
| 67.4 | 100 |
| 73.5 | 5-10 |

22. The process according to claim 16, wherein a reaction temperature is from 100° C. to 280° C.

23. The process according to claim 16, wherein a reaction pressure is equal to or greater than 10 bar.

24. The process according to claim 16, wherein the reaction is conducted in a solvent selected from the group consisting of water, ethanol, tetrahydrofuran and 1,4-dioxane.

25. The process according to claim 16, wherein a pH of the reaction is from 5.3 to 10.5.

26. The process according to claim 16, wherein the reaction is conducted continuously.

27. The process according to claim 16, wherein the reaction is conducted batchwise.

28. The process according to claim 16, wherein the catalyst system is obtained by a method comprising:
a) producing a mixture (i) comprising a ruthenium salt solution (ii) and an aqueous suspension of aluminum oxide (iii);
b) bringing the mixture (i) to a temperature of from greater than 0° C. to less than 100° C. and a pH of 0 to 14;
c) separating off the catalyst system by filtering the mixture (i) after treatment according to b); and
d) drying the separated catalyst system obtained in c).
